(19) United States
(12) Patent Application Publication
(10) Pub. No.: US 2025/0197451 A1
(43) Pub. Date: Jun. 19, 2025

(54) USE OF BIOACTIVE POLYPEPTIDE SYNTHESIZED ON BASIS OF KDM2B SEQUENCE IN NEURAL DIFFERENTIATION AND REGENERATION AND REPAIR OF MESENCHYMAL STEM CELLS

(71) Applicant: CAPITAL MEDICAL UNIVERSITY SCHOOL OF STOMATOLOGY, Beijing (CN)

(72) Inventors: Zhipeng FAN, Beijing (CN); Yangyang CAO, Beijing (CN); Chen ZHANG, Beijing (CN)

(73) Assignee: CAPITAL MEDICAL UNIVERSITY SCHOOL OF STOMATOLOGY, Beijing (CN)

(21) Appl. No.: 18/685,578

(22) PCT Filed: Aug. 30, 2021
(86) PCT No.: PCT/CN2021/115296

§ 371 Date: Sep. 16, 2024

(51) Int. Cl. C07K 14/00; A61K 38/00; A61P 25/28

(52) U.S. Cl. CPC C07K 14/001; A61P 25/28; A61K 38/00

(57) ABSTRACT

The present invention relates to a bioactive polypeptide synthesized on the basis of histone demethylase, and the use thereof in the neural differentiation process of mesenchymal stem cells. Disclosed in the present invention are possible protein-protein interaction binding sites of histone demethylase KDM2B and histone methylate EZH2, and disclosed are the roles of KDM2B and the bioactive polypeptide synthesized on the basis of the same in the neural differentiation process of mesenchymal stem cells and the tissue regeneration of injured spinal nerves. On this basis, it is concluded that KDM2B and the bioactive polypeptide synthesized on the basis of the same may play a role in promoting the neural differentiation of stem cells from apical papilla and the regeneration of injured spinal nerves.

### CROSS REFERENCE TO RELATED APPLICATIONS

This application is a National Phase Application under 35 U.S.C § 371 of International Application PCT/CN2021/115296, filed Aug. 30, 2021, which is hereby incorporated by reference in its entirety.

### INCORPORATION OF SEQUENCE LISTING

The Sequence Listing that is contained in the file named “OP2421-US-1385 Sequence Listing 2024 Jul. 9” (date created: Jul. 9, 2024; file size: 69,000 bytes) filed by electronic submission is incorporated by reference herein.

### FIELD

The present invention relates to the field of bioengineering technology, and in particular to a synthesized bioactive polypeptide on the basis of histone demethylase KDM2B and use in the differentiation of mesenchymal stem cells into neurons and in the regeneration repair of injured nervous tissues thereof.

### BACKGROUND

Nervous tissue widely distributed in the cranio-maxillofacial region is vulnerable to injury. In recent years, epidemiological investigations show that 55.2% of patients with cranio-maxillofacial trauma have varying degrees of nerve injury, 2.2% are also accompanied with spinal injury, and the neurological dysfunction in complications during subsequent treatment accounts for 24%. In the past, the treatment was mainly performed, for example, by neuroprotective measures such as hypothermia, by using neuroprotective drugs such as glucocorticoids and glutamate antagonists, and by stimulating the nerve to heal itself with stimulation such as electrical stimulation, heat stimulation, and mechanical stimulation. These treatment methods are complex and have a long period, a low recovery rate of neural function, and a high causing-disability rate. Autogenous/allogenic nerve transplantation has problems such as difficulty in obtaining donor tissue and secondary injury in donor site. Meanwhile, the transplanted nerves cannot remodel well, and still cannot recovery the morphology of the tissue structure and the damaged function. At present, it is still desirable to improve the effectiveness of the clinical therapy for nerve injury.

Biological regeneration based on mesenchymal stem cells (MSCs) will be a novel therapy for repairing the function of damaged nerves in the future. Due to the good differentiation ability in response to signals of tissue injury, MSCs can promote regeneration and repair of injured nerves by promoting the formation of cells for repairing nerves and the axonal regeneration. However, because neural-derived stem cells have shortcomings such as a limited source and difficult collection, so it is still necessary to find other ‘seed’ cells. Odontogenic MSCs are developed from the neural crest that is initially originates from the ectoderm and closely related to the origin of the nervous tissue, thus Odontogenic MSCs have the advantage of being more convenient for translational application. Wherein, stem cells from apical papilla (SCAPs) are found in the apical papilla at the apex of the tooth root and a study has shown that they can form pulpal nerve-like tissue in vivo, suggesting that SCAPs are the usable seed cells for the regeneration of nerves. Currently, the main reasons for the limited potential use of SCAPs are the low efficiency of neural differentiation and unclear regulatory mechanisms. Therefore, it is particularly important to effectively explore key regulatory targets and promote the use of the repair potential of odontogenic mesenchymal stem cells.

In recent studies, epigenetic regulation of chromatin was identified as a key mechanism determining neural lineage differentiation of MSCs. Genome-wide mapping for epigenetic regulatory shows significantly higher methylation scores at the sites 27th and 4th of lysine on histone H3 (H3K27 and H3K4) of differentiated MSCs. Methylation of histones occurs mostly on lysine residues (K) and is the main form of covalent modification of histones in epigenetic mechanisms. The groups with histone methylation present in the promoter region of a gene, for example H3K27me3, hinder the transcriptional expression of this gene, or for example H3K4me3, promote the transcriptional expression of this gene. The histone methylase/demethylase play a key regulatory role in the histone methylation, and the two have certain functional interactions, for example the formation of the complex by binding of functional domains.

A chromatin immunoprecipitation (ChIP)-sequencing of the whole genome of embryonic stem cells shows that the binding of histone to methylase EZH2 is highly correlated with the H3K27me3 modification of the promoter region of genes regulating neural differentiation. EZH2 is a core member of the polycomb groups (PcGs), and the H3K27me3 modified by the EZH2 belongs to a histone methylation modification that inhibits the transcription of a gene and is believed to hinder the differentiation of cells with specific fate. It is found that EZH2 inhibits the differentiation of the neural progenitor cells from neural stem cells, and the ability of the ventral midbrain-derived neural stem cells to differentiate into neuron is effectively improved by using the antagonist EPZ005687 specific against H3K27me3, suggesting a possible inhibitory effect of EZH2 on the differentiation of nerves. Therefore, it is critical to effectively regulate the function of EZH2.

It is found that the KDM2B is involved in recruiting the PRC2-EZH2 complex for anchoring of downstream target genes and subsequent covalent modification of histones, suggesting a possible cross between KDM2B and EZH2 in epigenetic regulation. KDM2B is a histone demethylase, and it is involved in the regulation of various cellular processes, mainly by demethylating the trimethylation at the site H3K4 of histone (H3K4me3). Some researchers found that KDM2B depends on its various functional domains such as JmjC, CxxC, and PHD, for function. However, the role of KDM2B in the neural differentiation of odontogenic MSCs is unclear and the possible interaction between KDM2B and EZH2 proteins is also unclear.

Currently, “polypeptide microarray assay”, is the emerging technical approach for analyzing protein binding, allowing researchers to follow a widely accepted viewpoint on the specific binding site of the protein-protein interaction. Specifically, based on the immune-binding of proteins, the protein of interest is hybridized to a microarray chip synthesized on the basis of the full-length amino acid sequence of the other protein, and the signals for this hybrid binding are amplified by using an enzyme-linked tag to identify a possible binding site of the protein to the other protein and to obtain the specific amino acid sequence of the fragment at that site. Thereby, the polypeptide microarray can be used to investigate the possible functional binding domains of KDM2B and EZH2. Understanding the binding process of these two proteins holds the promise of determining the key interaction sites of potential epigenetic modification enzymes that regulate the neural differentiation of odontogenic MSCs, which is contributed to the development of novel biological drugs useful in promoting the neural differentiation of odontogenic MSCs and to enhance the regeneration of injured nerves under clinical conditions.

The present invention aims to elucidate the role of KDM2B in neural differentiation and regeneration of odontogenic mesenchymal stem cells, to elucidate the possible interactions between KDM2B and EZH2, and to investigate the possible binding fragments of KDM2B and EZH2 by using a polypeptide microarray.

### SUMMARY

In view of the above, the present invention provides a synthesized bioactive polypeptide on the basis of histone demethylase KDM2B and use of the same in regulation of the differentiation of mesenchymal stem cells into neurons and in the regeneration and repair of injured nervous tissues thereof, aiming to solve the problem that the prior art does not involve the regulation in the neural differentiation of odontogenic mesenchymal stem cells and in the regeneration and repair of injured nervous tissues by the KDM2B gene and the bioactive polypeptide thereof.

In order to achieve the above objects, the present invention provides the following technical solutions.

The present invention provides use of KDM2B in the manufacture an agent or a drug for neural induction of stem cells from apical papilla in vitro, wherein the KDM2B is overexpressed.

In some specific embodiments of the present invention, the overexpression of the KDM2B is used in the manufacture of an agent or a drug for promoting the formation of BIII-TUBULIN-positive and NESTIN-positive neurospheres by the stem cells from apical papilla in vitro.

The present invention further provides use of KDM2B in the manufacture of an agent or a drug for promoting regeneration and repair of injured spinal nerves mediated by stem cells form apical papilla in vivo, wherein the KDM2B is overexpressed.

More importantly, the present invention provides a polypeptide, comprising:
(I) an amino acid sequence set forth in any of SEQ ID NO(s): 1-266,
(II) an amino acid sequence derived from the amino acid sequence set forth in (I) by substitution, deletion or addition of one or more amino acids and having the same functions as the amino acid sequence set forth in (I), or
(III) an amino acid sequence having more than 90% identity to the amino acid sequence set forth in (I) or (II);
wherein the “more amino acids” in the “one or more amino acids” is two, there, four or five amino acids.

Furthermore, the present invention further provides a nucleic acid molecule encoding the polypeptide.

The present invention further provides an expression vector comprising the nucleic acid molecule.

More importantly, the present invention provides use of the polypeptides, the nucleic acid molecules, or the expression vectors in the manufacture of an agent or a drug for inducing neural differentiation of stem cells form apical papilla in vitro directly or indirectly.

Also importantly, the present invention further provides use of the polypeptides, the nucleic acid molecules, or the expression vectors in the manufacture of an agent or a drug for directly or indirectly promoting regeneration and repair of injured spinal nerves mediated by stem cells from apical papilla in vivo.

In some specific embodiments of the present invention, binding sites of KDM2B to EZH2 comprise a positive binding site selected from the group consisting of peptide 5, peptide 46, peptide 47, peptide 122, peptide 123, peptide 131, peptide 132, peptide 139, peptide 142, peptide 151, peptide 152, peptide 153, peptide 231, peptide 232 and peptide 233.

Wherein the amino acid sequence of the positive binding site is set forth in SEQ ID NO: 5, SEQ ID NO: 46, SEQ ID NO: 47, SEQ ID NO: 122, SEQ ID NO: 123, SEQ ID NO: 131, SEQ ID NO: 132, SEQ ID NO: 139, SEQ ID NO: 142, SEQ ID NO: 151, SEQ ID NO: 152, SEQ ID NO: 153, SEQ ID NO: 231, SEQ ID NO: 232 or SEQ ID NO: 233.

The negative binding site is selected from the group consisting of peptide 83 and peptide 84, and the amino acid sequence of a negative binding site is set forth in SEQ ID NO: 267.

Furthermore, the present invention further provides a polypeptide comprising an amino acid sequence as set forth in any of SEQ ID NO(s): 267-270.

The present invention further provides a drug or an agent comprising the polypeptide and a pharmaceutically acceptable excipient.

Moreover, the present invention provides a method for investigating the KDM2B and for producing the polypeptide, comprising:
Step 1, culturing mesenchymal stem cells, constructing plasmids and transfecting with viral vector;
Step 2, the formation of βIII-TUBULIN, NESTIN double-positive neurospheres: inducing and culturing the mesenchymal stem cells using optimized neural stem cell medium for forming neurospheres, fixing the neurospheres with 4% paraformaldehyde on Day 9, after permeabilizing membranes with Triton, blocking the membrane with basic albumin solution, incubating the neurospheres with the specific primary antibody at 4° C. overnight, incubating the neurospheres sequentially with the fluorescent-labeled secondary antibodies, cytoskeleton dye Phalloidin and nuclei dye DAPI, and then stimulating the fluorescence for visualization, wherein the specific primary antibody is a polyclonal antibody against the neuron-specific βIII-TUBULIN and the NESTIN (a neural progenitor cell marker).
Step 3, transplantation of stem cells from apical papilla into rat model of spinal cord injury: transplanting about 1×10⁶ stem cells from apical papilla into 10-week-old rat at the site of a completely transected T10 spinal cord, after which evaluating the hind limb motor function of the rat by BBB score at weeks 0, 1, 2 and 3; obtaining spinal tissues at the injury site after 3 weeks, making paraffin sections, performing hematoxylin-eosin (HE) staining, and immunohistochemical staining for neuron-specific microtubulin βIII-TUBULIN and neurofilament-specific protein NEF-M for histopathological analysis;
particularly, transplanting stem cells from apical papilla into rat at the site of a completely transected T10 spinal cord, and slowly injecting 10 μl of stem cells from apical papilla at the center, left and right sides of the completely transected site by inserting the needle in the midline direction, respectively, with a 30 μl total injection system;
Step 4, co-immunoprecipitation: extracting total protein by lysing cells using RIPA lysate, incubating protein samples with the specific primary antibody and Protein A/G magnetic beads overnight at 4° C., washing with triethanolamine buffered saline (TBS), denaturing by boiling at 99° C. for Western blot, separating protein samples on a 10% SDS polyacrylamide gel, transferring to poly(vinylidene difluoride) (PVDF) membrane using a semi-dry transfer system, submerging the membrane in 5% skimmed milk, standing for 2 hours, incubating with the primary antibody overnight to form immune complex; and after incubating with rabbit or mouse immunoglobulin G, visualizing the immune complex using chemiluminescent substrate; wherein the primary antibodies are anti-KDM2B and anti-EZH2 polyclonal antibodies;
Step 5, polypeptide microarray-based immunohybridization and data analysis: designing an overlapping polypeptide library according to the full-length amino acid sequence of KDM2B, synthesizing the polypeptide microarray of KDM2B polypeptides by using a fully automated polypeptide microarray synthesizer, immunohybridizing the polypeptide microarray with recombinant proteins; activating the polypeptide microarray, blocking, incubating with biotin-labeled EZH2 protein with shaking overnight at 4° C., incubating with HRP, visualizing with the ECL substrate, imaging by using a Chempchemi digital imager, screening the colored points in the polypeptide microarray, by using the TotalLab image analysis software, analyzing the optical density of the colored points on the microarray image, and computing the color intensity of the colored points in percent using the “Spot Edge Average” algorithm;
Step 6: synthesis and purification of bioactive polypeptide: according to the obtained amino acid sequence of the bioactive polypeptide and sequence of cell-penetrating peptide, fluorescently labeling with FITC giving green fluorescence, sequentially synthesizing by adding the corresponding amino acids according to the amino acid sequence on the resins in dichloromethane solution, detecting with ninhydrin, capping with pyridine and acetic anhydride, washing, precipitating the crude product with ether, after centrifugation, purifying the crude product by liquid chromatography, freeze-drying by using a freeze-dryer, and obtaining the bioactive polypeptide powders.

The present invention further provides use of co-immunoprecipitation in the analysis of the role of the bioactive polypeptide provided by the invention on the binding of KDM2B to EZH2, use of the formation of βIII-TUBULIN, NESTIN double-positive neurospheres in the analysis of the role of the bioactive polypeptide on differentiation of neurospheres by stem cells from apical papilla, and use of local transplantation in the analysis of the role of the bioactive polypeptide on the regeneration of injured spinal nerves in rats.

The present invention provides bioactive polypeptides synthesized on the basis of histone demethylase KDM2B and use in the differentiation of mesenchymal stem cells into neurons and regeneration of injured spinal nerves thereof. By performing the following assays, including polypeptide microarray-based hybridization and data analysis, western blot, neurosphere formation and immunofluorescence staining of the βIII-TUBULIN and NESTIN double-positive neurospheres, and local transplantation in rat model of completely transected spinal cord, the effects of the KDM2B-based bioactive polypeptides on the differentiation of mesenchymal stem cells into neurons and regeneration of injured spinal nerves are identified. The present invention relates to the possible binding sites for the protein-protein interaction between histone demethylase KDM2B and histone methylase EZH2, the role of KDM2B and bioactive polypeptides in neuronal differentiation of mesenchymal stem cells, and the role of KDM2B and bioactive polypeptide in the regeneration of injured spinal nerves. It is shown that KDM2B and the bioactive polypeptides on the basis of KDM2B may play a role in promoting neural differentiation of stem cells from apical papilla and regeneration of injured spinal nerves.

### BRIEF DESCRIPTION OF DRAWINGS

In order to more clearly illustrate the examples of the present inventin or the technical solutions in the prior art, the following briefly introduces the drawings needed to be used in the description of the examples or the prior art.

FIG. 1 shows the expression of KDM2B in the mesenchymal stem cells and spinal cord tissue in examples of the present invention; wherein, FIG. 1(A) shows the result of real-time fluorescence reverse transcription quantitative PCR, indicating that the expression of KDM2B is increased during the neural induction of stem cells from apical papilla in vitro, with the highest expression occurring on the third day of induction; and FIGS. 1B and 1C show the results of immunohistochemical staining, indicating that the expression of KDM2B is reduced in injured spinal nerves compared to normal spinal nerves. GAPDH is used as an internal control and T-test is used to determine statistical significance in data. All error bars represent the SD (n=3). **P≤0.01; Red scale bar: 100 μm.

FIG. 2 is a schematic diagram showing that the overexpression of KDM2B provided in examples of the present invention promotes the formation of βIII-TUBULIN and NESTIN-positive neurospheres by stem cells from apical papilla in vitro; wherein, FIG. 2(A) shows the results of real-time fluorescence reverse transcription quantitative PCR; FIG. 2(B) shows the results of Western blotting, both indicating that the successful construction of stem cells from apical papilla overexpressing KDM2B; FIG. 2(C) shows that neurospheres formed by stem cells from apical papilla in the overexpressed-KDM2B group and the control vector group on Day 9 of induction in vitro, indicating that the overexpression of KDM2B promotes the formation of neurospheres by stem cells from apical papilla compared to the control vector group;

FIG. 2(D) and FIG. 2(E) show the results of immunofluorescence staining for βIII-TUBULIN-positive neurospheres and quantitative analysis thereof, respectively, indicating that overexpression of KDM2B promotes the formation of βIII-TUBULIN-positive neurospheres by stem cells from apical papilla in vitro; FIG. 2(F) and FIG. 2(G) show the results of immunofluorescence staining for NESTIN-positive neurospheres and quantitative analysis thereof, respectively, indicating that overexpression of KDM2B promotes the formation of NESTIN-positive neurospheres by stem cells from apical papilla in vitro; and FIG. 2(H-K) show the results of real-time fluorescence reverse transcription quantitative PCR, indicating that the overexpression of KDM2B up-regulates the expression of TH (H), NCAM (I), NEF (J), and NEUROD (K) in stem cells from apical papilla. GAPDH is used as an internal control and T-test is used to determine statistical significance in data. All error bars represent the SD (n=3); **P≤0.01; White scale bar: 100 μm.

FIG. 3 is a schematic diagram showing that the knockdown of KDM2B expression in examples of the present invention inhibits the formation of βIII-TUBULIN and NESTIN-positive neurospheres by stem cells from apical papilla in vitro; wherein, FIG. 3(A) shows the results of real-time fluorescence reverse transcription quantitative PCR, indicating the successful construction of stem cells from apical papilla with KDM2B knockdown; FIG. 3(B) shows that neurospheres formed by stem cells from apical papilla in the KDM2B knockdown group and the control Scramsh group on Day 9 of neural induction in vitro, indicating that the knockdown of KDM2B expression inhibits the formation of neurospheres by stem cells from apical papilla compared to the control Scramsh group; FIG. 3(C) and FIG. 3(D) show the results of immunofluorescence staining for βIII-TUBULIN-positive neurospheres and quantitative analysis thereof, respectively, indicating that knockdown of KDM2B expression inhibits the formation of βIII-TUBULIN-positive neurospheres by stem cells from apical papilla in vitro; and FIG. 3(E) and FIG. 3(F) show the results of immunofluorescence staining for NESTIN-positive neurospheres and quantitative analysis thereof, respectively, indicating that knockdown of KDM2B expression inhibits the formation of NESTIN-positive neurospheres by stem cells from apical papilla in vitro; GAPDH is used as an internal control and T-test is used to determine statistical significance in data. All error bars represent the SD (n=3); **P≤0.01; White scale bar: 100 μm.

FIG. 4 is a schematic diagram showing the overexpression of KDM2B in the examples of the present invention promotes the regeneration and repair of injured spinal nerves mediated by stem cells from apical papilla in vivo; wherein FIG. 4(A) shows the gross appearance of spinal nerves, indicating significant healing of injured tissues in the KDM2B overexpression group; FIG. 4(B) shows the results of the BBB scoring behavioral assessment, indicating that the overexpression of KDM2B significantly improves the hind limb motor function of rats; FIG. 4(C) shows the results of Hematoxylin-cosin (HE) staining, indicating that the overexpression of KDM2B significantly promotes the repair and regeneration of injured spinal nerve fibers; and FIG. 4(D) shows the results of immunohistochemical staining, indicating that the overexpression of KDM2B significantly up-regulates the expression of βIII-TUBULIN and NEF-M in the injured spinal nerves; the results shown in (A), (C) and (D) are obtained at the 3th week after the transplantation of stem cells from apical papilla. GAPDH is used as an internal control and T-test is used to determine statistical significance in data. All error bars represent the SD (n=3); *P≤0.05, **P≤0.01; White scale bar: 5 μm, Red scale bar: 100 μm.

FIG. 5 is a schematic diagram showing the result of the co-immunoprecipitation (CO-IP) assay in the examples of the present invention for detecting the binding of KDM2B to EZH2.

FIG. 6 is a schematic diagram showing polypeptide microarray containing EZH2 and KDM2B in the examples of the present invention; wherein FIG. 6(A) shows the colored points after hybridization; FIG. 6(B) shows the color intensity of the colored points indicating the binding sites on polypeptide microarray; FIG. 6(C) is a schematic diagram showing the fragment of the functional domain of EZH2 binding to KDM2B; and FIG. 6(D) shows the results of co-immunoprecipitation (CO-IP), indicating that 10 μg/ml bioactive polypeptides (peptide 46-47 (PPI group), peptide 122-123 (PP2 group) and peptide 131-132 (PP3 group)) efficiently block the binding of EZH2 to KDM2B.

FIG. 7 is a schematic diagram showing the bioactive peptide 46-47 (PPI group), peptide 122-123 (PP2 group) and peptide 131-132 (PP3 group) promote the formation of βIII-TUBULIN and NESTIN-positive neurospheres by stem cells from apical papilla in vitro; wherein FIG. 7(A) shows the neurospheres formed by stem cells from apical papilla on Day 9 of neural induction in vitro, indicating that peptide 46-47, peptide 122-123 and peptide 131-132 all significantly promote the formation of neurospheres by stem cells from apical papilla compared to the control ConPP group; FIG. 7(B) and 7 (C) show the results of immunofluorescence staining for βIII-TUBULIN and the corresponding quantification; and FIG. 7(D) and 7 (E) show the results of immunofluorescence staining for NESTIN and the corresponding quantification; these results shows that peptide 46-47, peptide 122-123 and peptide 131-132 all significantly promote the formation of βIII-TUBULIN-positive and NESTIN-positive neurospheres by stem cells from apical papilla in vitro compared to the control ConPP group. T-test is used to determine statistical significance in data. All error bars represent the SD (n=3); **P≤0.01; White scale bar: 5 mm.

FIG. 8 is a schematic diagram showing the bioactive peptide 46-47 (PPI group) promote the regeneration and repair of injured spinal nerves. In the rats transplanted with stem cells from apical papilla pre-treated with 10 μg/ml of peptide 46-47, the gross appearance of spinal tissues is shown in FIG. 8(A), indicating that injured neural tissue is significantly healed in the experimental group transplanted with cells pre-treated with 10 μg/ml of peptide 46-47; and the results of the BBB scoring for behavioral assessment are shown in FIG. 8(B), indicating that the hind limb motor function of rats is significantly improved in the experimental group transplanted with cells pre-treated with 10 μg/ml of peptide 46-47. In the rats injected with 10 μg/ml peptide 46-47 alone at the local site of injured spinal cord for 4 weeks, the gross view of spinal tissues is shown in FIG. 8(C), indicating that injured neural tissue is significantly healed in the experimental group injected with 10 μg/ml of peptide 46-47; and the results of BBB scoring for behavioral assessment is shown in FIG. 8(D), indicating that the hind limb motor function of rats is significantly improved in the experimental group injected with 10 μg/ml of peptide 46-47. FIG. 8(E) shows the results of joint analysis of BBB score, indicating that at 4th week of intervention, both the transplantation group with stem cells pretreated with 10 μg/ml of peptide 46-47 and the injection group with 10 μg/ml of peptide 46-47 alone significantly improve rat the hind limb motor function of rats, and there was no significant difference between these two groups. T-test is used to determine statistical significance in data. All error bars represent the SD (n=3); *P≤0.05, **P≤0.01; White scale bar: 5 mm.

FIG. 9 shows a schematic diagram of the polypeptide points on the membrane of the polypeptide microarray of KDM2B (left) and an image of Coomassie stained polypeptide microarray (right).

### DETAILED DESCRIPTION

The present invention discloses bioactive polypeptides on the basis of histone demethylase and use the same in the differentiation of mesenchymal stem cells into neurons. Those skilled in the art can learn from the disclosure and appropriately improve the process parameters. It should be noted that all similar replacements and modifications are obvious to those skilled in the art, and they are deemed to be included in the present invention. The method and the application of the present invention have been described through the preferred examples, and it is obvious that the method and application described herein may be changed or appropriately modified and combined to realize and apply the technology of the present invention by those skilled in the art without departing from the content, spirit and scope of the present invention.

An object of the present invention is to provide a bioactive polypeptide on the basis of histone demethylase KDM2B and to regulate the differentiation of mesenchymal stem cells into neurons by the same, aiming at solving the problem that the prior art does not involve the regulation by the KDM2B and bioactive polypeptide thereof in the differentiation of mesenchymal stem cells into neurons.

The present invention is achieved by providing a bioactive polypeptide on the basis of histone demethylase KDM2B and regulating the differentiation of mesenchymal stem cells into neurons by the bioactive polypeptide. The method for designing, analyzing polypeptide microarray of KDM2B, and synthesizing a bioactive polypeptide comprises the following steps:
Step 1, the design and synthesis of polypeptide microarray of KDM2B protein: obtaining the full-length sequence of human KDM2B protein by searching on the Uniprot website providing protein information (https://www.uniprot.org/), designing the polypeptide microarray by overlapping method according to the amino acid sequence of KDM2B consisting of 1336 amino acids, i.e., counting from the first amino acid, using the next 15 amino acids in length as the first overlapping peptide to design the first peptide on the microarray, performing a offset in a length of 5 amino acids relative to the 15th amino acid to reach a site, using the sequence of the next 15 amino acids in length behind the site as the second overlapping peptide to design the second peptide on the microarray, performing this process in a sequential extension, finally obtaining 266 peptides for the polypeptide microarray based on KDM2B protein.
Step 2, synthesis of polypeptide microarray: after activating, placing the matrix membrane of the microarray on a fully automated polypeptide microarray synthesizer, reacting the membrane with the solution of Fmoc (9-fluorenylmethoxycarbonyl)-amino acids automatically transferred to specific positions on the activated membrane according to the set procedure; sequentially immersing the membrane into the BSA Protein Scaling Solution I and II to block side chain, washing the membrane with dimethyl formamide (DMF) to remove the Fmoc protecting group at the amino-terminal, drying with ethanol; repeating the above steps until the full synthesis of polypeptide microarray, then removing the protective groups of the side chain with specific organic reagents, washing the membrane with CH₂Cl₂, washing with ethanol, drying, and using immediately or storing at −20° C.
Step 3, immune-hybridization of polypeptide microarray with recombinant protein: after activating the polypeptide microarray, adding the blocking solution, shaking at room temperature for 4 hours, washing the polypeptide microarray, labeling the EZH2 protein (a reaction solution with the concentration of 1.5 mg/ml) using EZ-link NHS-PEO4-Biotinylation kit (prod #21455), after diluting to a final concentration of with the blocking solution, mixing the reaction solution of biotin-labelled EZH2 protein samples (final concentration: 1 μg/ml) with the polypeptide microarray, incubating with shaking at 4° C. overnight, meanwhile, incubating the control with the blocking solution, diluting the Streptavidin-HRP (High Sensitivity Streptavidin-HRP (prod #21133)) with the blocking solution at a ratio of 1:10000, incubating the polypeptide microarray with 5 ml of diluted the Streptavidin-HRP with shaking at room temperature for 2 hours, washing the polypeptide microarray, and visualizing by using ECL reagents on a Chempchemi digital imager.
Step 4, scan and data analysis: imaging the colored polypeptide microarray at 425 nm using the Chempchemi Imaging System with 200s exposure, analyzing the optical density of colored points using TotalLab image analysis software, and computing the optical density of each colored point using the optical density of the respective surrounding background as a reference for background correction using the “Spot Edge Average” algorithm in the software.
Step 5, synthesis of bioactive polypeptides: in the present invention, a total of 7 bioactive polypeptides are synthesized (amino acid sequences are detailed in Table 17); the sequence (YGRKKRRQRRR, SEQ ID NO: 286) of the cell-penetrating peptide is added to the amino-terminal (left terminal) of each bioactive polypeptide to facilitate it entering into the cells through the cell membrane and playing a role; each bioactive polypeptide is labeled with FITC giving green fluorescent; and each bioactive polypeptide is synthesized from the carboxyl terminal to the amino terminal.

Specifically, the synthesis of the bioactive polypeptide comprises: weighing resin (n equivalent) into a reactor, adding dichloromethane (DCM) for swelling for half an hour, removing the DCM, adding the first amino acid in the sequence (2n equivalents), adding diisopropylethylamine (DIEA, 2n equivalents), adding the appropriate amount of DMF and DCM (the appropriate amount refers to the amount to be added which is sufficient to allow the resin to be mixed sufficiently), in the presence of DIEA, DMF, and DCM, reacting under nitrogen bubbling for 60 minutes; adding methanol (about 5n equivalents), reacting for half an hour, removing the reaction solution, and washing with DMF and MEOH; adding the second amino acid in the sequence (also 2n equivalents), 1-[bis(dimethylamino)methyl]-1H-benzotriazolium-3-oxide hexafluorophosphate (HBTU, 2n equivalents), and DIEA to the reactor, reacting under nitrogen bubbling for 30 minutes, washing away the liquid, detecting with ninhydrin, end-capping with pyridine and acetic anhydride, washing, adding the appropriate amount of decapping solution to remove Fmoc (9-fluorenylmethoxycarbonyl) protecting group, washing, and detecting with ninhydrin; repeating the above steps, sequentially adding the subsequent amino acids in the sequence and performing the modification; and blow-drying the resin with nitrogen, removing the reaction column, pouring the resin into the flask, adding the cleavage solution (95% TFA, 2% ethanedithiol, 2% triisopropylsilane and 1% water) into the flask at an approximately ratio of the cleavage solution to the resin of 10 (ml): 1 (g), shaking, filtering off the resin, obtaining the filtrate, adding a large amount of ether to the filtrate, precipitating the crude product, centrifuging, washing, and obtaining the crude product of the bioactive polypeptide.
Step 6, purification and lyophilization of the bioactive polypeptide: purifying the crude product to the required purity by the High Performance Liquid Chromatography, placing purified liquid in a freeze-dryer for concentrating and freeze-drying, and finally obtaining a bioactive polypeptide as a yellowish powder.

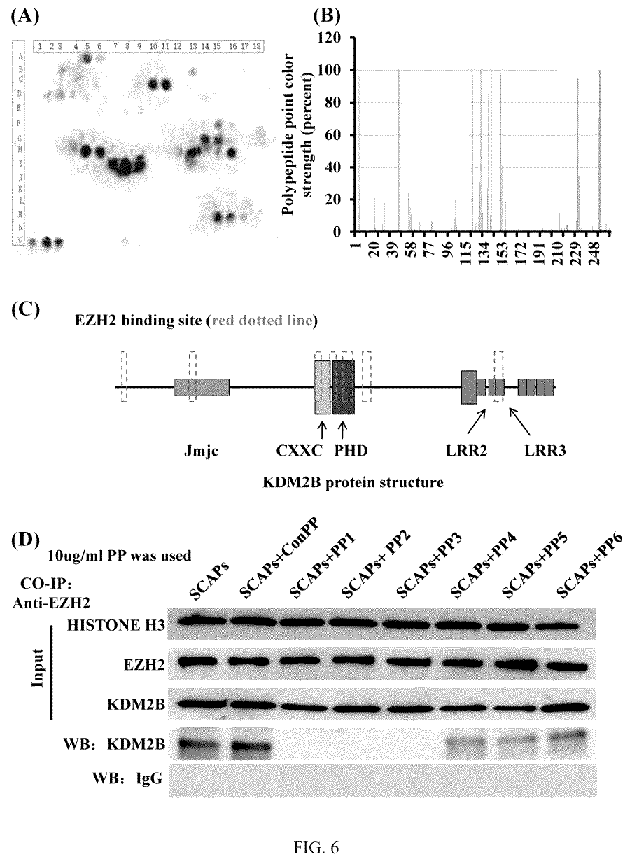

As shown in FIG. 6, a method for designing a polypeptide microarray of KDM2B, synthesizing and analyzing a bioactive polypeptide on the basis of KDM2B of the example of the present invention comprises the following steps:
S601, design and synthesis of polypeptide microarray of KDM2B protein Obtaining the full-length sequence of humanized KDM2B protein by searching on the Uniprot website providing protein information (https://www.uniprot.org/), designing the polypeptide microarray by overlapping method according to the amino acid sequence of KDM2B consisting of 1336 amino acids, i.e., counting from the first amino acid, using the next 15 amino acids in length as the first overlapping peptide to design the first polypeptide on the microarray, performing a offset in a length of 5 amino acids relative to the 15th amino acid to reach a site, using the sequence of the next 15 amino acids in length behind the site as the second overlapping peptide to design the second peptide on the microarray, performing this process in a sequential extension, finally obtaining 266 peptides (Table 12) for the polypeptide microarray based on KDM2B protein.
S602, synthesis of polypeptide microarray

After activating, placing the matrix membrane of the microarray on a fully automated polypeptide microarray synthesizer, reacting the membrane with the solution of Fmoc (9-fluorenylmethoxycarbonyl)-amino acids automatically transferred to specific positions on the activated membrane according to the set procedure; sequentially immersing the membrane into the BSA Protein Sealing Solution I and II to block side chain, washing the membrane with dimethyl formamide (DMF) to remove the Fmoc protecting group at the amino-terminal, drying with ethanol; repeating the above steps until the full synthesis of polypeptide microarray, then removing the protective groups of the side chain with specific organic reagents, washing the membrane with CH₂Cl₂, washing with ethanol, drying, and using immediately or storing at −20° C.
S603, immune-hybridization of polypeptide microarray with recombinant protein

Adding the blocking solution the polypeptide microarray, shaking at room temperature for 4 hours, washing the polypeptide microarray, labeling the EZH2 protein (a reaction solution with the concentration of 1.5 mg/ml) using EZ-link NHS-PEO4-Biotinylation kit (prod #21455), after diluting to a final concentration of with the blocking solution, incubating 5 ml of the reaction solution of biotin-labelled EZH2 protein samples (final concentration: 1 μg/ml) with the polypeptide microarray shaking at 4° C. overnight, meanwhile, incubating the control with the blocking solution, diluting the Streptavidin-HRP (High Sensitivity Streptavidin-HRP (prod #21133)) with the blocking solution at a ratio of 1:10000, incubating the polypeptide microarray with 5 ml of diluted the Streptavidin-HRP with shaking at room temperature for 2 hours, and visualizing by using ECL reagents.
S604, scan and data analysis of colored points

Imaging the polypeptide microarray at 425 nm using the Chempchemi Imaging System with 200s exposure, analyzing the optical density of colored points using TotalLab image analysis software, computing the color intensity of each colored point in percent by using the “Spot Edge Average” algorithm in the software.
S605, synthesis of bioactive polypeptide

In the present invention, a total of 7 bioactive polypeptides are synthesized (amino acid sequences are detailed in Table 17). Wherein, the sequence (YGRKKRRQRRR, SEQ ID NO: 286) of the cell-penetrating peptide is added to the amino-terminal (left terminal) of each bioactive polypeptide to facilitate it entering into the cell through the cell membrane and playing a role. In addition, each bioactive polypeptide is labeled with FITC giving green fluorescent. Each bioactive polypeptide is synthesized from the carboxyl terminal to the amino terminal.

Specifically, the synthesis of the bioactive polypeptide comprises: weighing resin (n equivalent) into a reactor, adding dichloromethane (DCM) for swelling for half an hour, removing the DCM, adding the first amino acid in the sequence (2n equivalents), adding diisopropylethylamine (DIEA, 2n equivalents), adding the appropriate amount of DMF and DCM (the appropriate amount refers to the amount to be added which is sufficient to allow the resin to be mixed sufficiently), in the presence of DIEA, DMF, and DCM, reacting under nitrogen bubbling for 60 minutes; adding methanol (about 5n equivalents), reacting for half an hour, removing the reaction solution, and washing with DMF and MEOH; adding the second amino acid in the sequence (also 2n equivalents), 1-[bis(dimethylamino)methyl]-1H-benzotriazolium-3-oxide hexafluorophosphate (HBTU, 2n equivalents), and DIEA to the reactor, reacting under nitrogen bubbling for 30 minutes, washing away the liquid, detecting with ninhydrin, end-capping with pyridine and acetic anhydride, washing, adding the appropriate amount of decapping solution to remove Fmoc (9-fluorenylmethoxycarbonyl) protecting group, washing, and detecting with ninhydrin; repeating the above steps, sequentially adding the subsequent amino acids in the sequence and performing the modification; and blow-drying the resin with nitrogen, removing the reaction column, pouring the resin into the flask, adding the cleavage solution (95% TFA, 2% ethanedithiol, 2% triisopropylsilane and 1% water) into the flask at an approximately ratio of the cleavage solution to the resin of 10 (ml): 1 (g), shaking, filtering off the resin, obtaining the filtrate, adding a large amount of ether to the filtrate, precipitating the crude product, centrifuging, washing, and obtaining the crude product of the bioactive polypeptide.
S606, purification and lyophilization of the bioactive polypeptide

Purifying the crude product to the required purity by the High Performance Liquid Chromatography, placing purified product in a freeze-dryer for concentrating and freeze-drying, and finally obtaining a yellowish bioactive polypeptide powder.
S607, handling of bioactive polypeptide

Dissolving bioactive polypeptide in stem cell culture solution (e.g. a α-MEM matrix solution containing 15% fetal bovine serum, 2 mmol/L glutamine, 100 U/ml penicillin and 100 μg/ml streptomycin used in the present invention) or cell culture phosphate-buffered saline to the storage concentration of 10 μg/μl, and dispensing and storing at −80° C. to avoid repeated freezing and thawing. Upon using, adding to the corresponding culture solution (e.g. the culture solution for neural induction of stem cells used in the present invention) with a 10 μg/ml of working concentration, preparing for immediate use.

The specific examples described herein are only used to explain the present invention rather than intended to limit the present invention.

In summary, the present invention investigates the role of KDM2B and a bioactive polypeptide from KDM2B in the neural differentiation of stem cells from apical papilla and in the regeneration and repair of injured neural tissues by using bioactive polypeptides based on KDM2B.

The data corresponding to the drawings in this application is shown in the following tables.

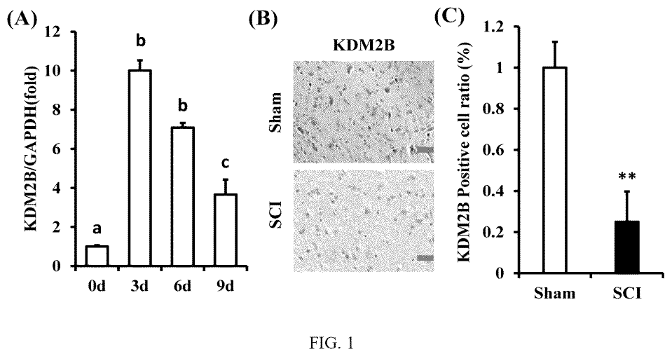

TABLE 1
Data of FIG. 1(A)
Expression of KDM2B/GAPDH during induction of neural
differentiation (fold)

| Group | 0 d | 3 d | 6 d | 9 d |
|---|---|---|---|---|
| Sample 1 | 1.06 | 9.77 | 7.14 | 3.07 |
| Sample 2 | 1.02 | 10.61 | 7.29 | 3.40 |
| Sample 3 | 0.93 | 9.65 | 6.83 | 4.53 |
| Means | 1.0016 | 10.0104 | 7.0877 | 3.6654 |
| SD | 0.0678 | 0.5241 | 0.2373 | 0.7658 |
| P Value (adjust 3 d) | 0.0005 | — | — | — |
| P Value (adjust 6 d) | 0.0002 | 0.0024 | — | — |
| P Value (adjust 9 d) | 0.0156 | 0.0049 | 0.0131 | — |

TABLE 2
Data of FIG. 1(C)
Positive cells expressing KDM2B in injured spinal cord (%)

| Group | Sham | SCI |
|---|---|---|
| Sample 1 | 61 | 15 |
| Sample 2 | 58 | 19 |
| Sample 3 | 54 | 17 |
| Sample 4 | 56 | 15 |
| Sample 5 | 60 | 20 |
| Means | 57.8 | 17.2 |
| SD | 2.8636 | 2.2804 |

| | |
|---|---|
| P Value | 0.000006 |

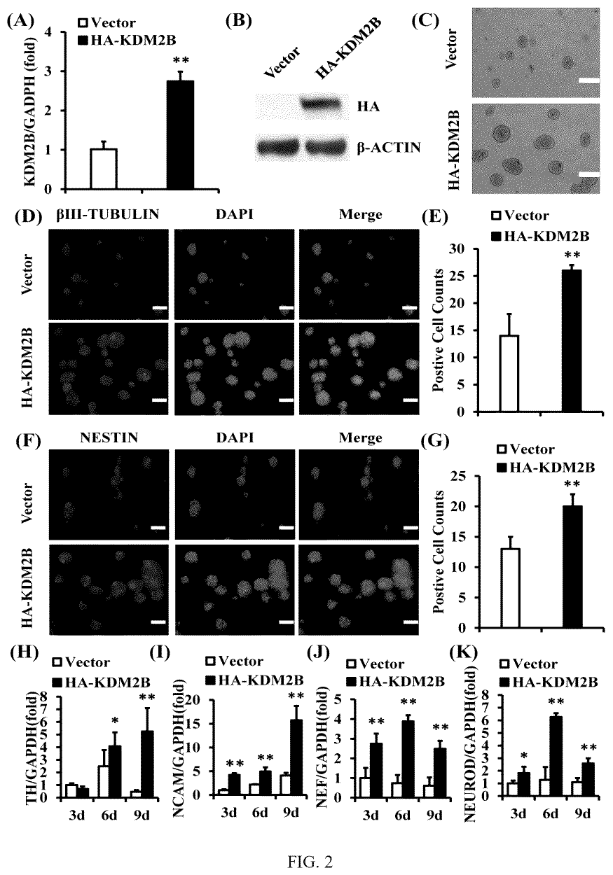

TABLE 3
Data of FIG. 2(A)
Expression of KDM2B/GAPDH in KDM2B-overexpressed SCAPs (fold)

| Group | Vector | HA-KDM2B |
|---|---|---|
| Sample 1 | 0.79 | 2.74 |
| Sample 2 | 1.07 | 3.00 |
| Sample 3 | 1.18 | 2.50 |
| Means | 1.0138 | 2.7458 |
| SD | 0.1982 | 0.2472 |

| | |
|---|---|
| P Value | 0.0068 |

TABLE 4
Data of FIG. 2(E)
Number of βIII-TUBULI-positive neurospheres

| Group | Vector | HA-KDM2B |
|---|---|---|
| Sample 1 | 11 | 20 |
| Sample 2 | 9 | 23 |
| Sample 3 | 6 | 22 |
| Sample 4 | 11 | 19 |
| Sample 5 | 8 | 19 |
| Means | 9 | 20.6 |
| SD | 2.1213 | 1.8166 |

| | |
|---|---|
| p Value | 0.0008 |

TABLE 5
Data of FIG. 2(G)
Number of NESTIN-positive neurospheres

| Group | Vector | HA-KDM2B |
|---|---|---|
| Sample 1 | 11 | 28 |
| Sample 2 | 12 | 26 |
| Sample 3 | 13 | 27 |
| Sample 4 | 10 | 25 |
| Sample 5 | 9 | 24 |
| Means | 11 | 26 |
| SD | 1.5811 | 1.5811 |

| | |
|---|---|
| p Value | 0.00000529 |

TABLE 6
Data of FIG. 2(H-K)
Expression of TH/GAPDH, NCAM/GAPDH, NEF/GAPDH, and NEUROD/GAPDH during
induction of neural differentiation of SCAPs expressing HA-KDM2B (fold)

| | | Vector | HA-KDM2B | Vector | HA-KDM2B | Vector | HA-KDM2B | Vector | HA-KDM2B |
|---|---|---|---|---|---|---|---|---|---|
| Day | Group | TH | | NCAM | | NEF | | NEUROD | |
| 3 d | Means | 1 | 0.6894 | 1 | 0.0998 | 1 | 2.7448 | 1 | 1.8206 |
| 6 d | | 2.4857 | 4.0707 | 0.8442 | 0.0813 | 0.74 | 3.8795 | 1.2769 | 6.2591 |
| 9 d | | 0.4603 | 5.2495 | 1.5448 | 0.3386 | 0.6118 | 2.4832 | 1.0968 | 2.5846 |
| 3 d | SD | 0.1213 | 0.1351 | 0.1351 | 0.1214 | 0.5113 | 0.5011 | 0.5135 | 0.5121 |
| 6 d | | 1.3012 | 1.4123 | 0.1235 | 0.1114 | 0.4123 | 0.3111 | 0.4102 | 0.3211 |
| 9 d | | 0.1934 | 1.9034 | 0.2113 | 0.0923 | 0.4211 | 0.4092 | 0.4211 | 0.4191 |

| | | | | | |
|---|---|---|---|---|---|
| 3 d | p Value | 0.0685 | 0.0096 | 0.0036 | 0.0952 |
| 6 d | | 0.0481 | 0.0065 | 0.0059 | 0.0034 |
| 9 d | | 0.0098 | 0.0093 | 0.0036 | 0.0096 |

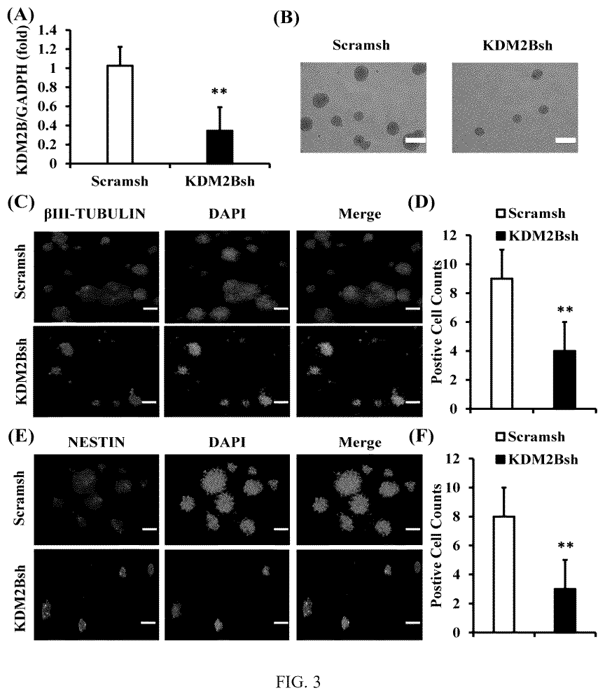

TABLE 7
Data of FIG. 3(A)
Expression of KDM2B/GAPDH in KDM2B-knockdown SCAPs (fold)

| Group | Scramsh | KDM2Bsh |
|---|---|---|
| Sample 1 | 1.03 | 0.34 |
| Sample 2 | 0.98 | 0.31 |
| Sample 3 | 0.99 | 0.28 |
| Means | 1.0003 | 0.3067 |
| SD | 0.0292 | 0.0294 |

| | |
|---|---|
| P Value | 0.0002 |

TABLE 8
Data of FIG. 3(D)
Number of βIII-TUBULI positive neurospheres

| Group | Scramsh | KDM2Bsh |
|---|---|---|
| Sample 1 | 10 | 4 |
| Sample 2 | 11 | 2 |
| Sample 3 | 9 | 6 |
| Sample 4 | 7 | 3 |
| Sample 5 | 8 | 4 |
| Means | 9 | 3.8 |
| SD | 1.5811 | 1.4832 |

| | |
|---|---|
| p Value | 0.0041 |

TABLE 9
Data of FIG. 3(F)
Number of NESTIN-positive neurospheres

| Group | Scramsh | KDM2Bsh |
|---|---|---|
| Sample 1 | 6 | 4 |
| Sample 2 | 9 | 2 |
| Sample 3 | 10 | 3 |
| Sample 4 | 9 | 2 |
| Sample 5 | 7 | 5 |
| Means | 8.2 | 3.2 |
| SD | 1.6432 | 1.3038 |

| | |
|---|---|
| p Value | 0.0075 |

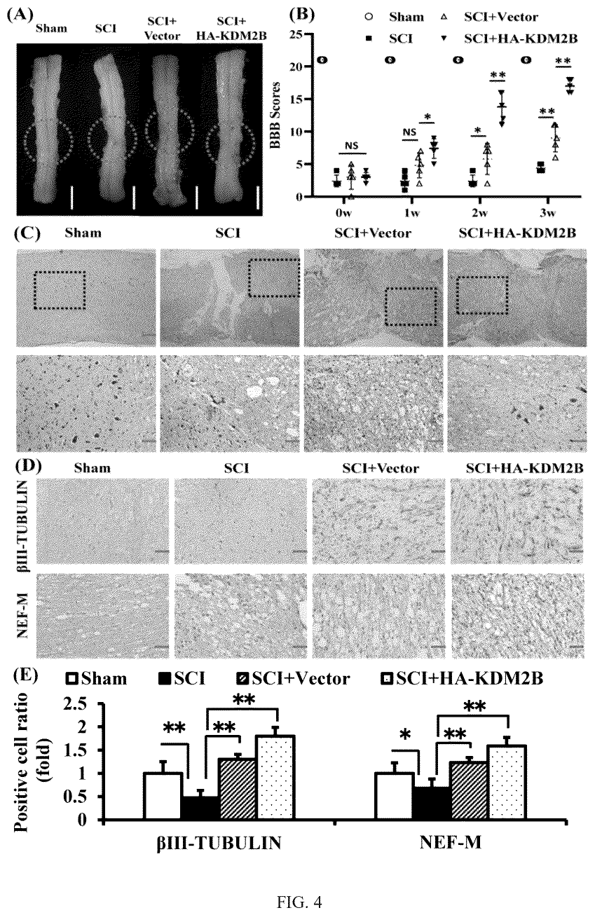

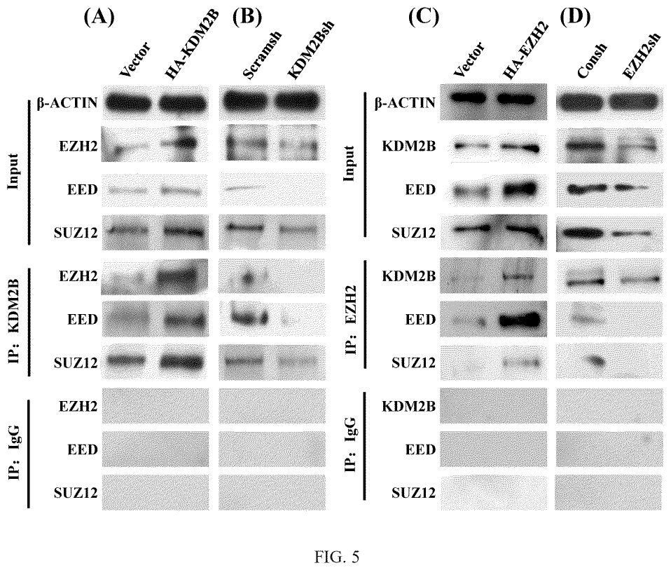

TABLE 10
Data of FIG. 4(B)
BBB score of hind limb motor function in rat model of spinal cord injury

| Time | | Sham | SCI | SCI + Vector | SCI + HA-KDM2B |
|---|---|---|---|---|---|
| point | Group | BBB score | | | |
| 0 w | Sample 1 | 21 | 2 | 0 | 4 |
| 0 w | Sample 2 | 21 | 2 | 3 | 3 |
| 0 w | Sample 3 | 21 | 2 | 4 | 2 |
| 0 w | Sample 4 | 21 | 2 | 5 | 3 |
| 0 w | Sample 5 | 21 | 4 | 3 | 3 |
| 1 w | Sample 1 | 21 | 1 | 2 | 5 |
| 1 w | Sample 2 | 21 | 2 | 5 | 8 |
| 1 w | Sample 3 | 21 | 2 | 6 | 9 |
| 1 w | Sample 4 | 21 | 3 | 7 | 7 |
| 1 w | Sample 5 | 21 | 4 | 4 | 8 |
| 2 w | Sample 1 | 21 | 2 | 2 | 11 |
| 2 w | Sample 2 | 21 | 2 | 5 | 16 |
| 2 w | Sample 3 | 21 | 2 | 8 | 12 |
| 2 w | Sample 4 | 21 | 2 | 7 | 16 |
| 2 w | Sample 5 | 21 | 4 | 7 | 14 |
| 3 w | Sample 1 | 21 | 4 | 6 | 16 |
| 3 w | Sample 2 | 21 | 5 | 9 | 18 |
| 3 w | Sample 3 | 21 | 4 | 11 | 18 |
| 3 w | Sample 4 | 21 | 4 | 8 | 17 |
| 3 w | Sample 5 | 21 | 5 | 11 | 16 |

TABLE 11
Data of FIG. 4(E)
Positive cells expressing indication in spinal cord (fold)

| | |
|---|---|
| βIII-TUBULIN | NEF-M |

| | | |
|---|---|---|
| Group | Indication | Relative fold |

| | | | |
|---|---|---|---|
| Sham | Means | 1 | 1 |
| SCI | | 0.47 | 0.68 |
| SCI + Vector | | 1.3 | 1.23 |
| SCI + HA-KDM2B | | 1.8 | 1.589 |
| Sham | SD | 0.250716 | 0.227296 |
| SCI | | 0.164719 | 0.197283 |
| Vector | | 0.10809 | 0.106517 |
| HA-KDM2B | | 0.191226 | 0.184728 |

TABLE 12
Data of FIG. 6(B)
Gray intensity (%) of colored points and the detailed sequence of 266 peptides
on the KDM2B polypeptide microarray

| No. of peptide | SEQ ID NO: | Amino acid sequence | Intensity (%) | Remark |
|---|---|---|---|---|
| 1 | SEQ ID NO: 1 | M A G P Q M G G S A E D H P P | 0.25 | |
| 2 | SEQ ID NO: 2 | M G G S A E D H P P R K R H A | 0 | noise |
| 3 | SEQ ID NO: 3 | E D H P P R K R H A A E K Q K | 0 | |
| 4 | SEQ ID NO: 4 | R K R H A A E K Q K K K T V I | 0 | |
| 5 | SEQ ID NO: 5 | A E K Q K K K T V I Y T K C F | 96.96 | |
| 6 | SEQ ID NO: 6 | K K T V I Y T K C F E F E S A | 27.53 | |
| 7 | SEQ ID NO: 7 | Y T K C F E F E S A T Q R P I | 0 | |
| 8 | SEQ ID NO: 8 | E F E S A T Q R P I D R Q R Y | 0 | noise |
| 9 | SEQ ID NO: 9 | T Q R P I D R Q R Y D E N E D | 0 | |
| 10 | SEQ ID NO: 10 | D R Q R Y D E N E D L S D V E | 0 | |
| 11 | SEQ ID NO: 11 | D E N E D L S D V E E I V S V | 0 | |
| 12 | SEQ ID NO: 12 | L S D V E E I V S V R G F S L | 0.13 | |
| 13 | SEQ ID NO: 13 | E I V S V R G F S L E E K L R | 0.05 | noise |
| 14 | SEQ ID NO: 14 | R G F S L E E K L R S Q L Y Q | 0.07 | noise |
| 15 | SEQ ID NO: 15 | E E K L R S Q L Y Q G D F V H | 0 | |
| 16 | SEQ ID NO: 16 | S Q L Y Q G D F V H A M E G K | 1.87 | |
| 17 | SEQ ID NO: 17 | G D F V H A M E G K D F N Y E | 0 | |
| 18 | SEQ ID NO: 18 | A M E G K D F N Y E Y V Q R E | 0 | noise |
| 19 | SEQ ID NO: 19 | D F N Y E Y V Q R E A L R V P | 0 | |
| 20 | SEQ ID NO: 20 | Y V Q R E A L R V P L I F R E | 1.28 | |
| 21 | SEQ ID NO: 21 | A L R V P L I F R E K D G L G | 21.08 | |
| 22 | SEQ ID NO: 22 | L I F R E K D G L G I K M P D | 2.54 | |
| 23 | SEQ ID NO: 23 | K D G L G I K M P D P D F T V | 0 | |
| 24 | SEQ ID NO: 24 | I K M P D P D F T V R D V K L | 0 | |
| 25 | SEQ ID NO: 25 | P D F T V R D V K L L V G S R | 0.02 | |
| 26 | SEQ ID NO: 26 | R D V K L L V G S R R L V D V | 0.36 | |
| 27 | SEQ ID NO: 27 | L V G S R R L V D V M D V N T | 0 | |
| 28 | SEQ ID NO: 28 | R L V D V M D V N T Q K G T E | 0 | |
| 29 | SEQ ID NO: 29 | M D V N T Q K G T E M S M S Q | 5.32 | |
| 30 | SEQ ID NO: 30 | Q K G T E M S M S Q F V R Y Y | 1.2 | |
| 31 | SEQ ID NO: 31 | M S M S Q F V R Y Y E T P E A | 19.19 | |
| 32 | SEQ ID NO: 32 | F V R Y Y E T P E A Q R D K L | 0 | noise |
| 33 | SEQ ID NO: 33 | E T P E A Q R D K L Y N V I S | 0 | noise |
| 34 | SEQ ID NO: 34 | Q R D K L Y N V I S L E F S H | 0 | |
| 35 | SEQ ID NO: 35 | Y N V I S L E F S H T K L E H | 5.95 | |
| 36 | SEQ ID NO: 36 | L E F S H T K L E H L V K R P | 0.89 | |
| 37 | SEQ ID NO: 37 | T K L E H L V K R P T V V D L | 0.73 | |
| 38 | SEQ ID NO: 38 | L V K R P T V V D L V D W V D | 0 | |
| 39 | SEQ ID NO: 39 | T V V D L V D W V D N M W P Q | 0.27 | |
| 40 | SEQ ID NO: 40 | V D W V D N M W P Q H L K E K | 0 | |
| 41 | SEQ ID NO: 41 | N M W P Q H L K E K Q T E A T | 0 | |
| 42 | SEQ ID NO: 42 | H L K E K Q T E A T N A I A E | 2.03 | |
| 43 | SEQ ID NO: 43 | Q T E A T N A I A E M K Y P K | 0 | |
| 44 | SEQ ID NO: 44 | N A I A E M K Y P K V K K Y C | 0 | noise |
| 45 | SEQ ID NO: 45 | M K Y P K V K K Y C L M S V K | 7.77 | |
| 46 | SEQ ID NO: 46 | V K K Y C L M S V K G C F T D | 100 | |
| 47 | SEQ ID NO: 47 | L M S V K G C F T D F H I D F | 100 | |
| 48 | SEQ ID NO: 48 | G C F T D F H I D F G G T S V | 0 | |
| 49 | SEQ ID NO: 49 | F H I D F G G T S V W Y H V F | 0 | |
| 50 | SEQ ID NO: 50 | G G T S V W Y H V F R G G K I | 0 | noise |
| 51 | SEQ ID NO: 51 | W Y H V E R G G K I F W L I P | 0 | |
| 52 | SEQ ID NO: 52 | R G G K I F W L I P P T L H N | 0.55 | |
| 53 | SEQ ID NO: 53 | F W L I P P T L H N L A L Y E | 0.7 | |
| 54 | SEQ ID NO: 54 | P T L H N L A L Y E E W V L S | 0 | noise |
| 55 | SEQ ID NO: 55 | L A L Y E E W V L S G K Q S D | 0 | |
| 56 | SEQ ID NO: 56 | E W V L S G K Q S D I F L G D | 24.62 | |
| 57 | SEQ ID NO: 57 | G K Q S D I F L G D R V E R C | 39.95 | |
| 58 | SEQ ID NO: 58 | I F L G D R V E R C Q R I E L | 15.62 | |
| 59 | SEQ ID NO: 59 | R V E R C Q R I E L K Q G Y T | 11.42 | |
| 60 | SEQ ID NO: 60 | Q R I E L K Q G Y T F F I P S | 0.3 | |
| 61 | SEQ ID NO: 61 | K Q G Y T F F I P S G W I H A | 0.85 | |
| 62 | SEQ ID NO: 62 | F F I P S G W I H A V Y T P V | 2.54 | |
| 63 | SEQ ID NO: 63 | G W I H A V Y T P V D S L V F | 1.74 | |
| 64 | SEQ ID NO: 64 | V Y T P V D S L V F G G N I L | 0 | spike |
| 65 | SEQ ID NO: 65 | D S L V F G G N I L H S F N V | 0 | spike |
| 66 | SEQ ID NO: 66 | G G N I L H S F N V P M Q L R | 0.54 | |
| 67 | SEQ ID NO: 67 | H S F N V P M Q L R I Y E I E | 0 | |
| 68 | SEQ ID NO: 68 | P M Q L R I Y E I E D R T R V | 6.33 | |
| 69 | SEQ ID NO: 69 | I Y E I E D R T R V Q P K F R | 0.15 | |
| 70 | SEQ ID NO: 70 | D R T R V Q P K F R Y P F Y Y | 0.02 | noise |
| 71 | SEQ ID NO: 71 | Q P K F R Y P F Y Y E M C W Y | 0.57 | |
| 72 | SEQ ID NO: 72 | Y P F Y Y E M C W Y V L E R Y | 0.04 | |
| 73 | SEQ ID NO: 73 | E M C W Y V L E R Y V Y C V T | 0.19 | noise |
| 74 | SEQ ID NO: 74 | V L E R Y V Y C V T Q R S H L | 0.25 | |
| 75 | SEQ ID NO: 75 | V Y C V T Q R S H L T Q E Y Q | 0 | |
| 76 | SEQ ID NO: 76 | Q R S H L T Q E Y Q R E S M L | 0 | |
| 77 | SEQ ID NO: 77 | T Q E Y Q R E S M L I D A P R | 0 | noise |
| 78 | SEQ ID NO: 78 | R E S M L I D A P R K P S I D | 1.74 | |
| 79 | SEQ ID NO: 79 | I D A P R K P S I D G F S S D | 1.13 | |
| 80 | SEQ ID NO: 80 | K P S I D G F S S D S W L E M | 6.61 | |
| 81 | SEQ ID NO: 81 | G F S S D S W L E M E E E A C | 7.19 | |
| 82 | SEQ ID NO: 82 | S W L E M E E E A C D Q Q P Q | 0 | |
| 83 | SEQ ID NO: 83 | E E E A C D Q Q P Q E E E E K | 0 | |
| 84 | SEQ ID NO: 84 | D Q Q P Q E E E E K D E E G E | 0 | |
| 85 | SEQ ID NO: 85 | E E E E K D E E G E G R D R A | 0 | |
| 86 | SEQ ID NO: 86 | D E E G E G R D R A P K P P T | 0.03 | |
| 87 | SEQ ID NO: 87 | G R D R A P K P P T D G S T S | 0 | |
| 88 | SEQ ID NO: 88 | P K P P T D G S T S P T S T P | 0 | |
| 89 | SEQ ID NO: 89 | D G S T S P T S T P S E D Q E | 0 | noise |
| 90 | SEQ ID NO: 90 | P T S T P S E D Q E A L G K K | 0.19 | noise |
| 91 | SEQ ID NO: 91 | S E D Q E A L G K K P K A P A | 0 | noise |
| 92 | SEQ ID NO: 92 | A L G K K P K A P A L R F L K | 0 | noise |
| 93 | SEQ ID NO: 93 | P K A P A L R F L K R T L S N | 0 | |
| 94 | SEQ ID NO: 94 | L R F L K R T L S N E S E E S | 0 | |
| 95 | SEQ ID NO: 95 | R T L S N E S E E S V K S T T | 0 | |
| 96 | SEQ ID NO: 96 | E S E E S V K S T T L A V D Y | 0 | noise |
| 97 | SEQ ID NO: 97 | V K S T T L A V D Y P K T P T | 0 | |
| 98 | SEQ ID NO: 98 | L A V D Y P K T P T G S P A T | 0.77 | |
| 99 | SEQ ID NO: 99 | P K T P T G S P A T E V S A K | 0 | noise |
| 100 | SEQ ID NO: 100 | G S P A T E V S A K W T H L T | 0 | |
| 101 | SEQ ID NO: 101 | E V S A K W T H L T E F E L K | 2.69 | |
| 102 | SEQ ID NO: 102 | W T H L T E F E L K G L K A L | 0 | |
| 103 | SEQ ID NO: 103 | E F E L K G L K A L V E K L E | 4.44 | |
| 104 | SEQ ID NO: 104 | G L K A L V E K L E S L P E N | 8.01 | |
| 105 | SEQ ID NO: 105 | V E K L E S L P E N K K C V P | 20.27 | |
| 106 | SEQ ID NO: 106 | S L P E N K K C V P E G I E D | 3.77 | |
| 107 | SEQ ID NO: 107 | K K C V P E G I E D P Q A L L | 0 | |
| 108 | SEQ ID NO: 108 | E G I E D P Q A L L E G V K N | 0 | |
| 109 | SEQ ID NO: 109 | P Q A L L E G V K N V L K E H | 0 | |
| 110 | SEQ ID NO: 110 | E G V K N V L K E H A D D D P | 0 | |
| 111 | SEQ ID NO: 111 | V L K E H A D D D P S L A I T | 0.99 | |
| 112 | SEQ ID NO: 112 | A D D D P S L A I T G V P V V | 0 | |
| 113 | SEQ ID NO: 113 | S L A I T G V P V V T W P K K | 1.55 | |
| 114 | SEQ ID NO: 114 | G V P V V T W P K K T P K N R | 0 | |
| 115 | SEQ ID NO: 115 | T W P K K T P K N R A V G R P | 0 | |
| 116 | SEQ ID NO: 116 | T P K N R A V G R P K G K L G | 0 | |
| 117 | SEQ ID NO: 117 | A V G R P K G K L G P A S A V | 0 | |
| 118 | SEQ ID NO: 118 | K G K L G P A S A V K L A A N | 0 | |
| 119 | SEQ ID NO: 119 | P A S A V K L A A N R T T A G | 0 | |
| 120 | SEQ ID NO: 120 | K L A A N R T T A G A R R R R | 0 | |
| 121 | SEQ ID NO: 121 | R T T A G A R R R R T R C R K | 1.31 | |
| 122 | SEQ ID NO: 122 | A R R R R T R C R K C E A C L | 100 | |
| 123 | SEQ ID NO: 123 | T R C R K C E A C L R T E C G | 97.26 | |
| 124 | SEQ ID NO: 124 | C E A C L R T E C G E C H F C | 0 | |
| 125 | SEQ ID NO: 125 | R T E C G E C H F C K D M K K | 1.57 | |
| 126 | SEQ ID NO: 126 | E C H F C K D M K K F G G P G | 6.05 | |
| 127 | SEQ ID NO: 127 | K D M K K F G G P G R M K Q S | 0 | noise |
| 128 | SEQ ID NO: 128 | F G G P G R M K Q S C I M R Q | 0 | |
| 129 | SEQ ID NO: 129 | R M K Q S C I M R Q C I A P V | 9.37 | |
| 130 | SEQ ID NO: 130 | C I M R Q C I A P V L P H T A | 30.8 | |
| 131 | SEQ ID NO: 131 | C I A P V L P H T A V C L V C | 100 | |
| 132 | SEQ ID NO: 132 | L P H T A V C L V C G E A G K | 100 | |
| 133 | SEQ ID NO: 133 | V C L V C G E A G K E D T V E | 12 | |
| 134 | SEQ ID NO: 134 | G E A G K E D T V E E E E G K | 0 | |
| 135 | SEQ ID NO: 135 | E D T V E E E E G K F N L M L | 0 | spike |
| 136 | SEQ ID NO: 136 | E E E G K F N L M L M E C S I | 0 | |
| 137 | SEQ ID NO: 137 | F N L M L M E C S I C N E I I | 0.24 | |
| 138 | SEQ ID NO: 138 | M E C S I C N E I I H P G C L | 26.8 | |
| 139 | SEQ ID NO: 139 | C N E I I H P G C L K I K E S | 85.09 | |
| 140 | SEQ ID NO: 140 | H P G C L K I K E S E G V V N | 0 | |
| 141 | SEQ ID NO: 141 | K I K E S E G V V N D E L P N | 10.79 | |
| 142 | SEQ ID NO: 142 | E G V V N D E L P N C W E C P | 100 | |
| 143 | SEQ ID NO: 143 | D E L P N C W E C P K C N H A | 0 | |
| 144 | SEQ ID NO: 144 | C W E C P K C N H A G K T G K | 0 | |
| 145 | SEQ ID NO: 145 | K C N H A G K T G K Q K R G P | 0 | noise |
| 146 | SEQ ID NO: 146 | G K T G K Q K R G P G F K Y A | 0.01 | noise |
| 147 | SEQ ID NO: 147 | Q K R G P G F K Y A S N L P G | 0 | |
| 148 | SEQ ID NO: 148 | G F K Y A S N L P G S L L K E | 0 | |
| 149 | SEQ ID NO: 149 | S N L P G S L L K E Q K M N R | 0 | spike |
| 150 | SEQ ID NO: 150 | S L L K E Q K M N R D N K E G | 0 | spike |
| 151 | SEQ ID NO: 151 | Q K M N R D N K E G Q E P A K | 100 | |
| 152 | SEQ ID NO: 152 | D N K E G Q E P A K R R S E C | 97.79 | |
| 153 | SEQ ID NO: 153 | Q E P A K R R S E C E E A P R | 41.58 | |
| 154 | SEQ ID NO: 154 | R R S E C E E A P R R R S D E | 0 | spike |
| 155 | SEQ ID NO: 155 | E E A P R R R S D E H S K K V | 0 | noise |
| 156 | SEQ ID NO: 156 | R R S D E H S K K V P P D G L | 0 | |
| 157 | SEQ ID NO: 157 | H S K K V P P D G L L R R K S | 18.41 | |
| 158 | SEQ ID NO: 158 | P P D G L L R R K S D D V H L | 0 | |
| 159 | SEQ ID NO: 159 | L R R K S D D V H L R K K R K | 0 | |
| 160 | SEQ ID NO: 160 | D D V H L R K K R K Y E K P Q | 0 | spike |
| 161 | SEQ ID NO: 161 | R K K R K Y E K P Q E L S G R | 0 | |
| 162 | SEQ ID NO: 162 | Y E K P Q E L S G R K R A S S | 0 | |
| 163 | SEQ ID NO: 163 | E L S G R K R A S S L Q T S P | 0 | noise |
| 164 | SEQ ID NO: 164 | K R A S S L Q T S P G S S S H | 0.34 | noise |
| 165 | SEQ ID NO: 165 | L Q T S P G S S S H L S P R P | 0 | noise |
| 166 | SEQ ID NO: 166 | G S S S H L S P R P P L G S S | 0.84 | |
| 167 | SEQ ID NO: 167 | L S P R P P L G S S L S P W W | 0 | |
| 168 | SEQ ID NO: 168 | P L G S S L S P W W R S S L T | 0 | |
| 169 | SEQ ID NO: 169 | L S P W W R S S L T Y F Q Q Q | 0 | |
| 170 | SEQ ID NO: 170 | R S S L T Y F Q Q Q L K P G K | 2.26 | |
| 171 | SEQ ID NO: 171 | Y F Q Q Q L K P G K E D K L F | 0 | |
| 172 | SEQ ID NO: 172 | L K P G K E D K L F R K K R R | 0 | |
| 173 | SEQ ID NO: 173 | E D K L F R K K R R S W K N A | 0 | |
| 174 | SEQ ID NO: 174 | R K K R R S W K N A E D R M A | 0.47 | |
| 175 | SEQ ID NO: 175 | S W K N A E D R M A L A N K P | 0 | |
| 176 | SEQ ID NO: 176 | E D R M A L A N K P L R R F K | 0 | |
| 177 | SEQ ID NO: 177 | L A N K P L R R F K Q E P E D | 0 | |
| 178 | SEQ ID NO: 178 | L R R F K Q E P E D E L P E A | 0 | |
| 179 | SEQ ID NO: 179 | Q E P E D E L P E A P P K T R | 0.39 | |
| 180 | SEQ ID NO: 180 | E L P E A P P K T R E S D H S | 0 | |
| 181 | SEQ ID NO: 181 | P P K T R E S D H S R S S S P | 0.3 | noise |
| 182 | SEQ ID NO: 182 | E S D H S R S S S P T A G P S | 0.36 | noise |
| 183 | SEQ ID NO: 183 | R S S S P T A G P S T E G A E | 0 | noise |
| 184 | SEQ ID NO: 184 | T A G P S T E G A E G P E E K | 0 | noise |
| 185 | SEQ ID NO: 185 | T E G A E G P E E K K K V K M | 0 | |
| 186 | SEQ ID NO: 186 | G P E E K K K V K M R R K R R | 0 | |
| 187 | SEQ ID NO: 187 | K K V K M R R K R R L P N K E | 0.95 | |
| 188 | SEQ ID NO: 188 | R R K R R L P N K E L S R E L | 0.8 | |
| 189 | SEQ ID NO: 189 | L P N K E L S R E L S K E L N | 0 | |
| 190 | SEQ ID NO: 190 | L S R E L S K E L N H E I Q R | 0.26 | |
| 191 | SEQ ID NO: 191 | S K E L N H E I Q R T E N S L | 0 | |
| 192 | SEQ ID NO: 192 | H E I Q R T E N S L A N E N Q | 0.19 | |
| 193 | SEQ ID NO: 193 | T E N S L A N E N Q Q P I K S | 0 | |
| 194 | SEQ ID NO: 194 | A N E N Q Q P I K S E P E S E | 0 | |
| 195 | SEQ ID NO: 195 | Q P I K S E P E S E G E E P K | 2.29 | |
| 196 | SEQ ID NO: 196 | E P E S E G E E P K R P P G I | 1.04 | |
| 197 | SEQ ID NO: 197 | G E E P K R P P G I C E R P H | 0 | |
| 198 | SEQ ID NO: 198 | R P P G I C E R P H R F S K G | 0 | noise |
| 199 | SEQ ID NO: 199 | C E R P H R F S K G L N G T P | 0.18 | |
| 200 | SEQ ID NO: 200 | R F S K G L N G T P R E L R H | 0.91 | |
| 201 | SEQ ID NO: 201 | L N G T P R E L R H Q L G P S | 0.18 | noise |
| 202 | SEQ ID NO: 202 | R E L R H Q L G P S L R S P P | 0.64 | noise |
| 203 | SEQ ID NO: 203 | Q L G P S L R S P P R V I S R | 0 | |
| 204 | SEQ ID NO: 204 | L R S P P R V I S R P P P S V | 1.91 | |
| 205 | SEQ ID NO: 205 | R V I S R P P P S V S P P K C | 0.39 | noise |
| 206 | SEQ ID NO: 206 | P P P S V S P P K C I Q M E R | 1.29 | |
| 207 | SEQ ID NO: 207 | S P P K C I Q M E R H V I R P | 0 | |
| 208 | SEQ ID NO: 208 | I Q M E R H V I R P P P I S P | 0 | noise |
| 209 | SEQ ID NO: 209 | H V I R P P P I S P P P D S L | 0.32 | noise |
| 210 | SEQ ID NO: 210 | P P I S P P P D S L P L D D G | 0 | noise |
| 211 | SEQ ID NO: 211 | P P D S L P L D D G A A H V M | 0 | |
| 212 | SEQ ID NO: 212 | P L D D G A A H V M H R E V W | 0 | |
| 213 | SEQ ID NO: 213 | A A H V M H R E V W M A V F S | 11.8 | |
| 214 | SEQ ID NO: 214 | H R E V W M A V F S Y L S H Q | 0 | |
| 215 | SEQ ID NO: 215 | M A V F S Y L S H Q D L C V C | 2.82 | |
| 216 | SEQ ID NO: 216 | Y L S H Q D L C V C M R V C R | 0 | |
| 217 | SEQ ID NO: 217 | D L C V C M R V C R T W N R W | 0.19 | |
| 218 | SEQ ID NO: 218 | M R V C R T W N R W C C D K R | 2.93 | |
| 219 | SEQ ID NO: 219 | T W N R W C C D K R L W T R I | 0.56 | noise |
| 220 | SEQ ID NO: 220 | C C D K R L W T R I D L N H C | 4.32 | |
| 221 | SEQ ID NO: 221 | L W T R I D L N H C K S I T P | 4.09 | |
| 222 | SEQ ID NO: 222 | D L N H C K S I T P L M L S G | 0 | |
| 223 | SEQ ID NO: 223 | K S I T P L M L S G I I R R Q | 0 | noise |
| 224 | SEQ ID NO: 224 | L M L S G I I R R Q P V S L D | 0 | |
| 225 | SEQ ID NO: 225 | I I R R Q P V S L D L S W T N | 0 | noise |
| 226 | SEQ ID NO: 226 | P V S L D L S W T N I S K K Q | 0.51 | |
| 227 | SEQ ID NO: 227 | L S W T N I S K K Q L S W L I | 0 | noise |
| 228 | SEQ ID NO: 228 | I S K K Q L S W L I N R L P G | 0 | noise |
| 229 | SEQ ID NO: 229 | L S W L I N R L P G L R D L V | 0 | noise |
| 230 | SEQ ID NO: 230 | N R L P G L R D L V L S G C S | 7.03 | |
| 231 | SEQ ID NO: 231 | L R D L V L S G C S W I A V S | 100 | |
| 232 | SEQ ID NO: 232 | L S G C S W I A V S A L C S S | 95.43 | |
| 233 | SEQ ID NO: 233 | W I A V S A L C S S S C P L L | 34.8 | |
| 234 | SEQ ID NO: 234 | A L C S S S C P L L R T L D V | 3.59 | |
| 235 | SEQ ID NO: 235 | S C P L L R T L D V Q W V E G | 2.02 | |
| 236 | SEQ ID NO: 236 | R T L D V Q W V E G L K D A Q | 2.1 | |
| 237 | SEQ ID NO: 237 | Q W V E G L K D A Q M R D L L | 1.18 | |
| 238 | SEQ ID NO: 238 | L K D A Q M R D L L S P P T D | 0.62 | |
| 239 | SEQ ID NO: 239 | M R D L L S P P T D N R P G Q | 1 | |
| 240 | SEQ ID NO: 240 | S P P T D N R P G Q M D N R S | 0 | |
| 241 | SEQ ID NO: 241 | N R P G Q M D N R S K L R N I | 0 | noise |
| 242 | SEQ ID NO: 242 | M D N R S K L R N I V E L R L | 0.64 | |
| 243 | SEQ ID NO: 243 | K L R N I V E L R L A G L D I | 0.92 | |
| 244 | SEQ ID NO: 244 | V E L R L A G L D I T D A S L | 0.9 | |
| 245 | SEQ ID NO: 245 | A G L D I T D A S L R L I I R | 0.31 | |
| 246 | SEQ ID NO: 246 | T D A S L R L I I R H M P L L | 0 | noise |
| 247 | SEQ ID NO: 247 | R L I I R H M P L L S K L H L | 3.66 | |
| 248 | SEQ ID NO: 248 | H M P L L S K L H L S Y C N H | 1.15 | |
| 249 | SEQ ID NO: 249 | S K L H L S Y C N H V T D Q S | 0 | |
| 250 | SEQ ID NO: 250 | S Y C N H V T D Q S I N L L T | 0 | |
| 251 | SEQ ID NO: 251 | V T D Q S I N L L T A V G T T | 0 | |
| 252 | SEQ ID NO: 252 | I N L L T A V G T T T R D S L | 0.22 | |
| 253 | SEQ ID NO: 253 | A V G T T T R D S L T E I N L | 70.41 | |
| 254 | SEQ ID NO: 254 | T R D S L T E I N L S D C N K | 100 | |
| 255 | SEQ ID NO: 255 | T E I N L S D C N K V T D Q C | 100 | |
| 256 | SEQ ID NO: 256 | S D C N K V T D Q C L S F F K | 5.43 | |
| 257 | SEQ ID NO: 257 | V T D Q C L S F F K R C G N I | 0.23 | noise |
| 258 | SEQ ID NO: 258 | L S F F K R C G N I C H I D L | 0.72 | noise |
| 259 | SEQ ID NO: 259 | R C G N I C H I D L R Y C K Q | 1.36 | |
| 260 | SEQ ID NO: 260 | C H I D L R Y C K Q V T K E G | 22.3 | |
| 261 | SEQ ID NO: 261 | R Y C K Q V T K E G C E Q F I | 5.21 | |
| 262 | SEQ ID NO: 262 | V T K E G C E Q F I A E M S V | 0 | noise |
| 263 | SEQ ID NO: 263 | C E Q F I A E M S V S V Q F G | 0.05 | noise |
| 264 | SEQ ID NO: 264 | A E M S V S V Q F G Q V E E K | 2.91 | |
| 265 | SEQ ID NO: 265 | S V Q F G Q V E E K L L Q K L | 2.09 | |
| 266 | SEQ ID NO: 266 | V Q F G Q V E E K L L Q K L S | 0 | noise |

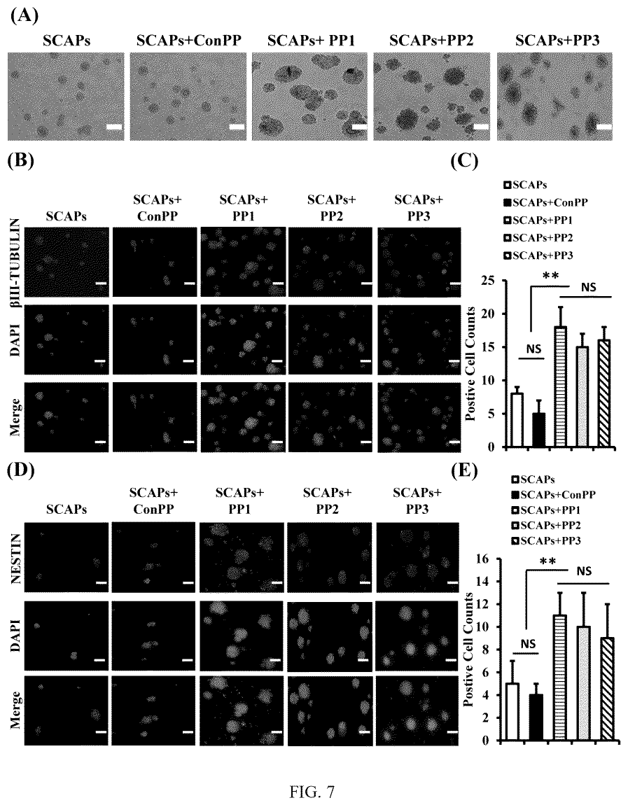

TABLE 13
Data of FIG. 7(C)
Number of βIII-TUBULIN-positive neurosphers

| Group | SCAPs | SCAPs + ConPP | SCAPs + PP1 | SCAPs + PP2 | SCAPs + PP3 |
|---|---|---|---|---|---|
| Sample 1 | 9 | 6 | 16 | 15 | 16 |
| Sample 2 | 9 | 7 | 17 | 13 | 14 |
| Sample 3 | 7 | 6 | 19 | 16 | 19 |
| Sample 4 | 8 | 8 | 20 | 18 | 14 |
| Sample 5 | 7 | 8 | 18 | 13 | 17 |
| Means | 8 | 7 | 18 | 15 | 16 |
| SD | 1 | 1 | 1.58113883 | 2.121320344 | 2.1213203 |
| p Value (adjust SCAPs) | — | 0.115100 | 0.000338 | 0.001539 | 0.001788 |
| p Value (adjust SCAPs + ConPP) | 0.115100 | — | 0.000032 | 0.000793 | 0.000913 |

TABLE 14
Data of FIG. 7(E)
Number of NESTIN-positive neurosphers

| Group | SCAPs | SCAPs + ConPP | SCAPs + PP1 | SCAPs + PP2 | SCAPs + PP3 |
|---|---|---|---|---|---|
| Sample 1 | 4 | 3 | 9 | 9 | 7 |
| Sample 2 | 4 | 4 | 12 | 11 | 8 |
| Sample 3 | 5 | 6 | 11 | 11 | 12 |
| Sample 4 | 7 | 4 | 10 | 9 | 11 |
| Sample 5 | 5 | 3 | 13 | 10 | 7 |
| Means | 5 | 4 | 11 | 10 | 9 |
| SD | 1.224745 | 1.224745 | 1.581139 | 1 | 2.345208 |
| p Value (adjust | — | 0.115100 | 0.001599 | 0.001970 | 0.004385 |
| SCAPs) | | | | | |
| p Value (adjust | 0.115100 | — | 0.000720 | 0.000089 | 0.000692 |
| SCAPs + ConPP) | | | | | |

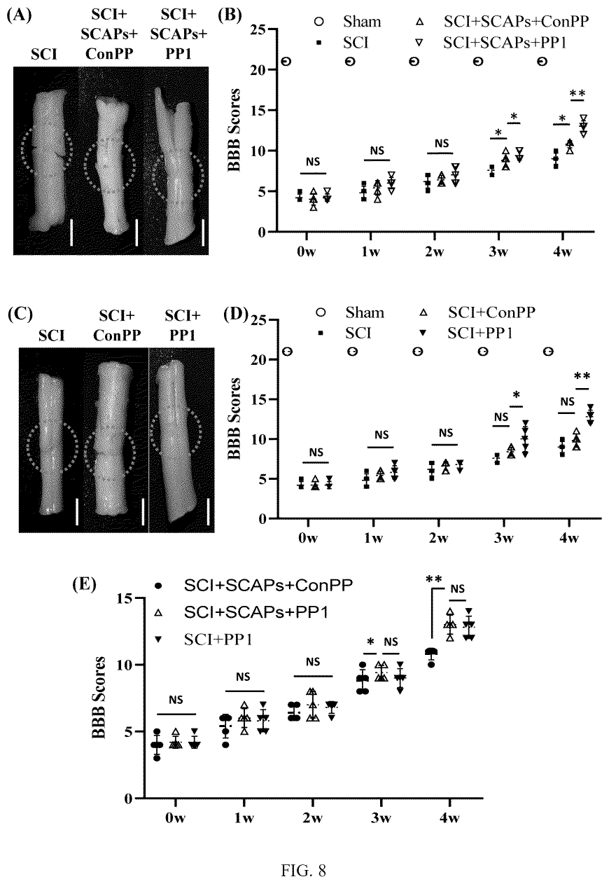

TABLE 15
Row Data of FIG. 8(B), (D), and (E)
BBB score of hind limb motor function in rat
model of spinal cord injury

| | | | | SCI + | SCI + | | |
|---|---|---|---|---|---|---|---|
| | | Sham | SCI | SCAPs + ConPP | SCAPs + PP1 | SCI + ConPP | SCI + PP1 |
| Week | Group | BBB score | | | | | |
| 0 w | Sample 1 | 21 | 4 | 4 | 4 | 5 | 4 |
| 0 w | Sample 2 | 21 | 4 | 5 | 4 | 4 | 4 |
| 0 w | Sample 3 | 21 | 4 | 4 | 5 | 4 | 5 |
| 0 w | Sample 4 | 21 | 4 | 4 | 4 | 4 | 4 |
| 0 w | Sample 5 | 21 | 5 | 4 | 4 | 5 | 4 |
| 1 w | Sample 1 | 21 | 4 | 5 | 5 | 6 | 6 |
| 1 w | Sample 2 | 21 | 4 | 5 | 6 | 6 | 6 |
| 1 w | Sample 3 | 21 | 5 | 5 | 5 | 6 | 6 |
| 1 w | Sample 4 | 21 | 5 | 6 | 7 | 5 | 5 |
| 1 w | Sample 5 | 21 | 6 | 6 | 6 | 5 | 7 |
| 2 w | Sample 1 | 21 | 5 | 6 | 7 | 7 | 6 |
| 2 w | Sample 2 | 21 | 6 | 7 | 7 | 6 | 7 |
| 2 w | Sample 3 | 21 | 7 | 7 | 6 | 7 | 8 |
| 2 w | Sample 4 | 21 | 6 | 6 | 7 | 6 | 6 |
| 2 w | Sample 5 | 21 | 7 | 7 | 7 | 8 | 8 |
| 3 w | Sample 1 | 21 | 7 | 8 | 9 | 9 | 9 |
| 3 w | Sample 2 | 21 | 7 | 8 | 10 | 9 | 9 |
| 3 w | Sample 3 | 21 | 8 | 8 | 8 | 10 | 10 |
| 3 w | Sample 4 | 21 | 8 | 9 | 9 | 8 | 9 |
| 3 w | Sample 5 | 21 | 8 | 9 | 9 | 9 | 10 |
| 4 w | Sample 1 | 21 | 9 | 10 | 12 | 12 | 12 |
| 4 w | Sample 2 | 21 | 8 | 11 | 14 | 11 | 13 |
| 4 w | Sample 3 | 21 | 9 | 10 | 13 | 11 | 13 |
| 4 w | Sample 4 | 21 | 10 | 12 | 12 | 11 | 14 |
| 4 w | Sample 5 | 21 | 9 | 13 | 13 | 12 | 13 |

The raw materials and reagents used in the synthesis and use of the bioactive polypeptide provided by the present invention are commercially available.

The present invention is further illustrated below in conjunction with examples.

### EXAMPLES

### Example 1 Cell Culture and Induction of Neural Differentiation In Vitro

All experimental involving stem cells in the present invention adhered to the guideline for human embryonic stem cell research, the use of human tissues was approved by the Ethics Committee of Capital Medical University, and volunteers signed informed consent prior to operation. Briefly, after the teeth was disinfected with 75% alcohol and washed 10 times continuously with phosphate buffer, the apical papilla tissue located at the apex of dental root was obtained by carefully dissecting with a sterile scalpel blade and cut with a sterile scissor. After which, 1 mL of collagenase I (Worthington Biochemical Corp., Lakewood, NJ) at a concentration of 3 mg/ml and 1 mL of dispase (Roche Diagnostics Corp., Indianapolis, IN) at a concentration of 4 mg/ml were added, and then the apical papilla tissue was placed at 37° C. with shaking for 1 hour of digestion, and filtered through a 70 μm filter (BD Biosciences, San Jose, CA) to obtain a single cell suspension. Next, these cells were seed in Minimum Eagle Medium (MEM) (Invitrogen, Carlsbad, CA), 15% fetal bovine serum, 2 mmol/L glutamine, 100 U/ml penicillin, and 100 mg/ml streptomycin were added, and the cells were placed in a humidified incubator with 5% CO₂ at 37° C. The medium was changed every 3 days.

The stem cells used were identified based on cell surface marker for stem cells by known method, and stem cells in passages 3-5 were used in subsequent experiments. For neural induction in vitro, stem cells were cultured and induced using optimized neural stem cell medium (Neurobasal A medium containing 2% B27, 40 ng/ml bFGF, 20 ng/ml EGF, 2 mM L-glutamine, 100 U/ml penicillin and 100 μg/ml streptomycin), which was changed every 3 days.

The observation was carried out under an inverted microscope on Day 9 of induction to preliminary determine the formation of neurospheres. For immunofluorescence staining, neurospheres were collected on Day 9 of induction, fixed in 4% paraformaldehyde, treated with Triton to permeabilize membranes, blocked with albumin solution, incubated with specific primary antibody at 4° C. overnight, incubated sequentially with fluorescence-labeled secondary antibody against specific species, PDI (cytoskeleton dye) and DAPI (nuclei dye), and visualized by stimulating the fluorescence. The specific primary antibodies (purchased from Abcam, Cambridge, USA) is polyclonal antibodies against the neuron-specific βIII-TUBULIN and the NESTIN.

### Example 2 Plasmid Construction of Plasmid and Transfection with Viral Vector

The plasmid was constructed according to standard methods. The complementary shRNA sequence targeting KDM2B gene was cloned into the viral vector pLKO.1 plasmid, sequenced and identified to complete the construction of the KDM2B shRNA plasmid. After PCR primers were designed for the full-length sequence of the KDM2B gene, the full-length sequence of the KDM2B gene was obtained by amplification, and the HA Tag was added. The tagged full-length KDM2B gene was ligated with the pQCXIN retroviral expression vector, sequenced and identified to obtain the plasmid containing the HA tagged full-length KDM2B gene. The virus packaging was carried out, and the resulting virus was collected, and the virus titer was measured. The virus was aliquoted and stored in the refrigerator at −80° C. The virus at a titer of 10-7 was incubated with stem cells from apical papillae in the presence of polybrene for 48 hours for infection, and the antibiotic was used to screen the infected cells. The sequence of the complementary shRNA targeting the KDM2B gene was represented as KDM2Bsh, with a nucleic acid sequence of 5′-ATTTTGACGGGGTGGATAATCTG-3′ (SEQ ID NO: 287).

### Example 3 Extraction of Total RNA, Reverse Transcription (RT) PCR, and Real-Time Fluorescence Reverse Transcription Quantitative PCR

Total RNA was extracted from three samples of stem cells from apical papilla derived from different donors and purified by using extraction reagents and RNA extraction kit (QIAGEN, GmBH, Germany), and dissolved in RNase-Free Water (QIAGEN). Total RNA was quantified by spectrophotometer ND-2100 (Thermo Fisher) and RNA integrity was assessed using Agilent 2100 (Agilent). For detecting mRNA of interest, equal amounts of cDNA samples were synthesized by reverse transcription using a random primer kit (QIAGEN) according to the instrument instruction (Invitrogen) and Real-time PCR were performed by using fluorescent PCR (QIAGEN) and the iCycler IQ Multicolor Real-Time PCR Detection System. Primers were designed using the online program Primer 3 and the sequences of primers were detailed in Table 18. GAPDH was used as an internal control, and relative levels of mRNA were calculated by using the 2^{-ΔΔCt} method.

### Example 4 Co-Immunoprecipitation and Western Blot

Three total protein samples were extracted from stem cells from apical papilla derived from different donors by lying cells using RIPA lysate. For co-immunoprecipitation, equal amount of total protein samples were incubated with the specific primary antibody and Protein A/G magnetic beads at 4° C. overnight, washed with triethanolamine buffered saline (TBS), and denatured by boiling at 99° C. for Western blot or stored at −80° C. for later use.

For Western blot assay, equal amount of three total protein samples were separated on 10% polyacrylamide gel by SDS-PAGE and transferred to poly(vinylidene difluoride) (PVDF) membrane by using a semi-dry transfer system. The membrane was submerged in 5% non-fat milk for 2 hours, incubated with the primary antibody overnight to form immune complex. After incubated with rabbit/mouse anti IgG antibody, the immune complex was visualized with chemiluminescent substrate. The primary antibodies are anti KDM2B and anti EZH2 polyclonal antibodies (Abcam).

### Example 5 Rat Model of Spinal Cord Injury and Transplantation of Stem Cells from Apical Papilla

The animal experiments had been approved by the Animal Care and Use Committee of the affiliated Beijing Stomatological Hospital of Capital Medical University. The stem cells from apical papilla were transplanted into 10-week-old rats at the T10 spinal cord segment with complete transection, about 1×10⁶ stem cells from apical papilla per rat. Particularly, the transplantation was carried out by slowly injecting each 10 μl of stem cells from apical papilla at the center, left and right sides of the complete transection site by inserting the needle in the midline direction, respectively, with a total injection system of 30 μl. In the control vector group and KDM2B overexpression group, the number of rats transplanted with stem cells from apical papilla is 5. These experiments were carried out according to the animal protocol. After transplantation, the hind limb motor function of the rat was evaluated using BBB score at weeks of 0, 1, 2 and 3.

For histopathological analysis, spinal tissues at the injury site were obtained after 3 weeks, fixed with 10% paraformaldehyde, embedded in paraffin, cut into section with a thickness of 5 μm, stained with hematoxylin-cosin (HE). For immunohistochemical staining, the 5 μm section was dewaxed and rehydrated, and the endogenous peroxidase was blocked. After which the section was incubated with the specific primary antibody at 4° C. overnight, followed by biotin-horseradish peroxidase and then visualized by color development with a 3,3-diaminobenzidine (DAB) substrate. The specific primary antibodies are polyclonal antibodies against βIII-TUBULIN and NEF-M (Abcam).

### Example 6 Design and Synthesis of Polypeptide Microarray

The full-length sequence of human KDM2B protein was obtained by searching on the Uniprot website providing protein information (https://www.uniprot.org/), and the polypeptide microarray was designed by overlapping method according to the amino acid sequence of full-length KDM2B. The polypeptide microarray was synthesized by using a fully automated polypeptide microarray synthesizer. After blocking, the polypeptide microarray was incubated with biotin-labeled target protein for hybridization, visualized with the chemical chromogenic substrate and imaged using a Chempchemi digital imager. The image of colored polypeptide microarray was analyzed for the optical density of the colored points. Particularly, the optical density of each colored point was calculated using the optical density of the respective surrounding background as a reference for background correction. The highest optical density value of the colored point on membrane was set to 100%, and the optical density value of the remaining colored points was converted into percentages relative to the highest value. The points with an optical density percentage of more than 30% on the polypeptide membrane and also with an optical density percentage of less than 30% on the negative membrane were defined as positive colored points.

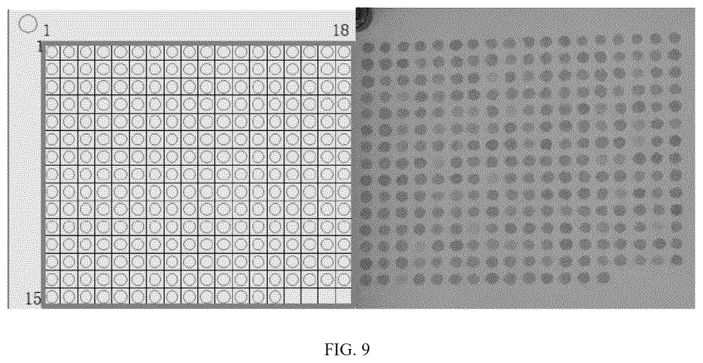

A schematic diagram of the polypeptide points on membrane of the KDM2B polypeptide microarray and the image of Coomassie-stained microarray are shown in FIG. 9.

As shown in the left panel of FIG. 9, the points containing a polypeptide on the microarray were arranged in 18 columns and 15 rows and numbered increasing from left to right and top to bottom, with a total of 266 points, i.e., 266 peptides. The 1-copy polypeptide microarray was synthesized according to the schematic diagram. The sequences of each peptide are detailed in Table 12. The right panel of FIG. 9 is the image of Coomassie-stained microarray after synthesis.

Further, KDM2B polypeptide microarray was hybridized with EZH2 protein.

For the hybridization experiment, a solution of NHS-PEO4-Biotinylation-labelled EZH2 protein (1 μg/ml) was used for the hybridization of the polypeptide microarray. Streptavidin-HRP was used to amplify the reaction signal according to the experimental procedure and conditions described above, and the time of color development was 5 minutes. The results are shown in FIG. 6(A), which shows that there is an obvious binding between the labeled-protein and some polypeptides on the microarray at the colored points.

Further, colored points on KDM2B polypeptide microarray was analyzed to determine the positive points.

The optical density data of the colored points were analyzed using TotalLab image analysis software. The highest optical density value of the colored point on the membrane was set to 100%, and the optical density value of the remaining colored points was converted into percentages relative to the highest value, and the resulting color intensity (grey percentage) of the colored points is shown in FIG. 6(B), where the abscissa is the numbering of points corresponding to the 266 peptides on the microarray membrane, and the ordinate is the optical density percentage. Based on experience, the points with an optical density percentage of more than 30% on the polypeptide membrane and also with an optical density percentage of less than 30% on the negative membrane were defined as positive colored points. The positive colored points and corresponding peptide sequence are shown in Table 16.

TABLE 16
Positive peptides on KDM2B peptide microarray after immune-hybridization
with EZH2 protein

| Number of points | SEQ ID NO: | Amino acid sequence |
|---|---|---|
| 5 | SEQ ID NO: 5 | A E K Q K K K T V I Y T K C F |
| 46 | SEQ ID NO: 46 | V K K Y C L M S V K G C F T D |
| 47 | SEQ ID NO: 47 | L M S V K G C F T D F H I D F |
| 122 | SEQ ID NO: 122 | A R R R R T R C R K C E A C L |
| 123 | SEQ ID NO: 123 | T R C R K C E A C L R T E C G |
| 131 | SEQ ID NO: 131 | C I A P V L P H T A V C L V C |
| 132 | SEQ ID NO: 132 | L P H T A V C L V C G E A G K |
| 139 | SEQ ID NO: 139 | C N E I I H P G C L K I K E S |
| 142 | SEQ ID NO: 142 | E G V V N D E L P N C W E C P |
| 151 | SEQ ID NO: 151 | Q K M N R D N K E G Q E P A K |
| 152 | SEQ ID NO: 152 | D N K E G Q E P A K R R S E C |
| 153 | SEQ ID NO: 153 | Q E P A K R R S E C E E A P R |
| 231 | SEQ ID NO: 231 | L R D L V L S G C S W I A V S |
| 232 | SEQ ID NO: 232 | L S G C S W I A V S A L C S S |
| 233 | SEQ ID NO: 233 | W I A V S A L C S S S C P L L |

### Example 7 Synthesis and Purification of Bioactive Polypeptide

The amino acid sequences of the bioactive polypeptides, cell-penetrating peptide and the fluorescent label with FITC giving green fluorescent were obtained. The corresponding amino acids were sequentially synthesized according to the amino acid sequence on swollen resin in dichloromethane solution, detected with ninhydrin, capped with pyridine and acetic anhydride, and washed. A crude product was precipitated using ether, centrifuged, purified by liquid chromatography, and freeze-dried by using a freeze-dryer to obtain the bioactive polypeptide as powders.

In order to further test the accuracy of the binding site of KDM2B to EZH2, 6 bioactive polypeptides were synthesized based on the positive binding sites reported in Table 16, while the negative binding sites (peptide 83-84) were randomly selected and synthesized as control polypeptide (ConPP group), which are shown in Table 17.

TABLE 17
Bioactive polypeptide synthesized on the basis of KDM2B

| | | Number of | |
|---|---|---|---|
| | Number of | Synthesized | |
| | synthesized | polypeptide | |
| Group | polypeptide | sequence | Amino acid sequence |
| ConPP group | peptide 83-84 | SEQ ID NO: 267 | EEEACDQQPQEEEEKDEEGE |
| PP1 group | peptide 46-47 | SEQ ID NO: 268 | VKKYCLMSVKGCFTDFHIDF |
| PP2 group | peptide 122-123 | SEQ ID NO: 269 | ARRRRTRCRKCEACLRTECG |
| PP3 group | peptide 131-132 | SEQ ID NO: 270 | CIAPVLPHTAVCLVCGEAGK |
| PP4 group | peptide 139-142 | SEQ ID NO: 271 | CNEIIHPGCLKIKESEGVVNDELPNCWECP |
| PP5 group | peptide 151-153 | SEQ ID NO: 272 | QKMNRDNKEGQEPAKRRSECEEAPR |
| PP6 group | peptide 231 | SEQ ID NO: 273 | LRDLVLSGCSWIAVSALCSSSCPLL |

Further, the co-immunoprecipitation results show that the addition of 10 μg/ml of peptide 46-47, peptide 122-123 and peptide 131-132 to stimulate apical papilla stem cells for 24 hours can significantly block the binding of KDM2B to EZH2 (FIG. 6(D)).

### Example 8 Use of Bioactive Polypeptide for Neural Differentiation of Stem Cells from Apical Papilla

Furthermore, 10 μg/ml tested peptides were added to the optimized neural stem cell medium. On Day 9 of induction of stem cells from apical papilla, peptide 46-47, peptide 122-123, and peptide 131-132 can significantly promote the formation of βIII-TUBULIN-positive (FIG. 7(B)) and NESTIN-positive neurospheres (FIG. 7(D)) by stem cells from apical papilla in vitro.

### Example 9 Use of the Bioactive Polypeptide in the Regeneration and Repair of Injured Neural Tissue

10 μg/ml of peptide 46-47 was selected and used to pre-treat stem cells from apical papilla for 24 hours, and then the pre-treated cells were immediately transplanted into 10-week-old rats at the complete transection site of T10 spinal cord (5 individual rats per group). After 4 weeks of transplantation, compared to the control group transplanted with cells pre-treated with ConPP, the gross appearance of spinal tissues shows that the injured neural tissue is significantly healed in the experimental group transplanted with cells pre-treated with 10 μg/ml of peptide 46-47 (FIG. 8(A)), and the BBB scoring for behavioral assessment shows that the hind limb motor function of rats is significantly improved in the experimental group transplanted with cells pre-treated with 10 μg/ml of peptide 46-47 (FIG. 8(B)).

10 μg/ml of peptide 46-47 in a total of 30 μl was weekly injected into 10-week-old rats at the complete transection site T10 spinal cord (5 individual rats per group). After 4 weeks, compared to the control group injected with ConPP alone, the gross appearance of spinal tissues shows that the injured neural tissue is significantly healed in the experimental group injected with 10 μg/ml of peptide 46-47 alone (FIG. 8(C)), and the BBB scoring for behavioral assessment shows that the hind limb motor function of rats is significantly improved in the experimental group injected with 10 μg/ml of peptide 46-47 alone (FIG. 8(D)).

Furthermore, the results of the BBB scoring for the transplantation group with stem cells pre-treated with control ConPP, transplantation group with stem cells pre-treated with 10 μg/ml of peptide 46-47, and injection group with 10 μg/ml of peptide 46-47 alone were subjected to joint analysis, showing that the hind limb motor function of rats is significantly improved in the transplantation group with stem cells pre-treated with 10 μg/ml of peptide 46-47 and injection group with 10 μg/ml of peptide 46-47 alone, with no significant difference between the two groups (FIG. 8(E)).

TABLE 18
Primers for real-time quantitative PCR

| Gene | Forward Primer | | Reverse Primer | |
|---|---|---|---|---|
| GAPDH | SEQ ID NO: 274 | CGGACCAATACGACCA | SEQ ID NO: 275 | AGCCACATCGCTCA |
| | | AATCCG | | GACACC |
| KDM2B | SEQ ID NO: 276 | TGTCCCAGTTTGTGCG | SEQ ID NO: 277 | GGACGCTTGACCA |
| | | TTACT | | AGTGCT |
| NCAM | SEQ ID NO: 278 | CGGGACCTGGAGGACT | SEQ ID NO: 279 | ACCATGTGCCCATC |
| | | TCTACCCG | | CAGAGTC |
| NEF | SEQ ID NO: 280 | CGAAGTCAATGGTTTC | SEQ ID NO: 281 | CCGCTCCTTCCCGT |
| | | CTCCACTTCG | | CCTACTAC |
| NEUROD | SEQ ID NO: 282 | CGACTGACCCCTACTC | SEQ ID NO: 283 | TGGAAGACATGGG |
| | | CTACCAGTCG | | AGCTGTCC |
| TH | SEQ ID NO: 284 | CCGAGCTGTGAAGGTG | SEQ ID NO: 285 | CGGGCCGGGTCTCT |
| | | TTTGA | | AGAT |

The bioactive polypeptide synthesized on the basis of histone demethylase KDM2B provided by the present invention and its use in the differentiation of mesenchymal stem cells into neurons and in the regeneration and repair of nerve injury are described in detail above. The principle and implementation of the present invention are illustrated by using specific embodiments herein. The above descriptions of the embodiments are only used to facilitate understanding of the method and the core idea of the present invention. It should be noted that, several improvements and modifications may be made by those skilled in the art to the present invention without departing from the principle of the present invention, and these improvements and modifications also fall within the protection scope of the claims of the present invention.

1-6. (canceled)

7. A method for inducing neural differentiation of stem cells from apical papilla in vitro, comprising contacting the stem cells from apical papilla with a polypeptide, wherein the polypeptide comprises an amino acid sequence selected from the group consisting of SEQ ID NO: 268, SEQ ID NO: 269 and SEQ ID NO: 270.

8. A method for promoting regeneration and repair of injured spinal nerves mediated by stem cells from apical papilla in vivo, comprising contacting the stem cells from apical papilla with a polypeptide, wherein the polypeptide comprises an amino acid sequence selected from the group consisting of SEQ ID NO: 268, SEQ ID NO: 269 and SEQ ID NO: 270.

9-10. (canceled)

11. The method according to claim 7, wherein the polypeptide comprises the amino acid sequence of SEQ ID NO: 268.

12. The method according to claim 8, wherein the polypeptide comprises the amino acid sequence of SEQ ID NO: 268.


SEQUENCE LISTING (287 sequences)

SEQ ID NO: 1 - protein, 15 aa, Artificial Sequence
  synthesized KDM2B peptide
MAGPQMGGSAEDHPP

SEQ ID NO: 2 - protein, 15 aa, Artificial Sequence
  synthesized KDM2B peptide
MGGSAEDHPPRKRHA

SEQ ID NO: 3 - protein, 15 aa, Artificial Sequence
  synthesized KDM2B peptide
EDHPPRKRHAAEKQK

SEQ ID NO: 4 - protein, 15 aa, Artificial Sequence
  synthesized KDM2B peptide
RKRHAAEKQKKKTVI

SEQ ID NO: 5 - protein, 15 aa, Artificial Sequence
  synthesized KDM2B peptide
AEKQKKKTVIYTKCF

SEQ ID NO: 6 - protein, 15 aa, Artificial Sequence
  synthesized KDM2B peptide
KKTVIYTKCFEFESA

SEQ ID NO: 7 - protein, 15 aa, Artificial Sequence
  synthesized KDM2B peptide
YTKCFEFESATQRPI

SEQ ID NO: 8 - protein, 15 aa, Artificial Sequence
  synthesized KDM2B peptide
EFESATQRPIDRQRY

SEQ ID NO: 9 - protein, 15 aa, Artificial Sequence
  synthesized KDM2B peptide
TQRPIDRQRYDENED

SEQ ID NO: 10 - protein, 15 aa, Artificial Sequence
  synthesized KDM2B peptide
DRQRYDENEDLSDVE

SEQ ID NO: 11 - protein, 15 aa, Artificial Sequence
  synthesized KDM2B peptide
DENEDLSDVEEIVSV

SEQ ID NO: 12 - protein, 15 aa, Artificial Sequence
  synthesized KDM2B peptide
LSDVEEIVSVRGFSL

SEQ ID NO: 13 - protein, 15 aa, Artificial Sequence
  synthesized KDM2B peptide
EIVSVRGFSLEEKLR

SEQ ID NO: 14 - protein, 15 aa, Artificial Sequence
  synthesized KDM2B peptide
RGFSLEEKLRSQLYQ

SEQ ID NO: 15 - protein, 15 aa, Artificial Sequence
  synthesized KDM2B peptide
EEKLRSQLYQGDFVH

SEQ ID NO: 16 - protein, 15 aa, Artificial Sequence
  synthesized KDM2B peptide
SQLYQGDFVHAMEGK

SEQ ID NO: 17 - protein, 15 aa, Artificial Sequence
  synthesized KDM2B peptide
GDFVHAMEGKDFNYE

SEQ ID NO: 18 - protein, 15 aa, Artificial Sequence
  synthesized KDM2B peptide
AMEGKDFNYEYVQRE

SEQ ID NO: 19 - protein, 15 aa, Artificial Sequence
  synthesized KDM2B peptide
DFNYEYVQREALRVP

SEQ ID NO: 20 - protein, 15 aa, Artificial Sequence
  synthesized KDM2B peptide
YVQREALRVPLIFRE

SEQ ID NO: 21 - protein, 15 aa, Artificial Sequence
  synthesized KDM2B peptide
ALRVPLIFREKDGLG

SEQ ID NO: 22 - protein, 15 aa, Artificial Sequence
  synthesized KDM2B peptide
LIFREKDGLGIKMPD

SEQ ID NO: 23 - protein, 15 aa, Artificial Sequence
  synthesized KDM2B peptide
KDGLGIKMPDPDFTV

SEQ ID NO: 24 - protein, 15 aa, Artificial Sequence
  synthesized KDM2B peptide
IKMPDPDFTVRDVKL

SEQ ID NO: 25 - protein, 15 aa, Artificial Sequence
  synthesized KDM2B peptide
PDFTVRDVKLLVGSR

SEQ ID NO: 26 - protein, 15 aa, Artificial Sequence
  synthesized KDM2B peptide
RDVKLLVGSRRLVDV

SEQ ID NO: 27 - protein, 15 aa, Artificial Sequence
  synthesized KDM2B peptide
LVGSRRLVDVMDVNT

SEQ ID NO: 28 - protein, 15 aa, Artificial Sequence
  synthesized KDM2B peptide
RLVDVMDVNTQKGTE

SEQ ID NO: 29 - protein, 15 aa, Artificial Sequence
  synthesized KDM2B peptide
MDVNTQKGTEMSMSQ

SEQ ID NO: 30 - protein, 15 aa, Artificial Sequence
  synthesized KDM2B peptide
QKGTEMSMSQFVRYY

SEQ ID NO: 31 - protein, 15 aa, Artificial Sequence
  synthesized KDM2B peptide
MSMSQFVRYYETPEA

SEQ ID NO: 32 - protein, 15 aa, Artificial Sequence
  synthesized KDM2B peptide
FVRYYETPEAQRDKL

SEQ ID NO: 33 - protein, 15 aa, Artificial Sequence
  synthesized KDM2B peptide
ETPEAQRDKLYNVIS

SEQ ID NO: 34 - protein, 15 aa, Artificial Sequence
  synthesized KDM2B peptide
QRDKLYNVISLEFSH

SEQ ID NO: 35 - protein, 15 aa, Artificial Sequence
  synthesized KDM2B peptide
YNVISLEFSHTKLEH

SEQ ID NO: 36 - protein, 15 aa, Artificial Sequence
  synthesized KDM2B peptide
LEFSHTKLEHLVKRP

SEQ ID NO: 37 - protein, 15 aa, Artificial Sequence
  synthesized KDM2B peptide
TKLEHLVKRPTVVDL

SEQ ID NO: 38 - protein, 15 aa, Artificial Sequence
  synthesized KDM2B peptide
LVKRPTVVDLVDWVD

SEQ ID NO: 39 - protein, 15 aa, Artificial Sequence
  synthesized KDM2B peptide
TVVDLVDWVDNMWPQ

SEQ ID NO: 40 - protein, 15 aa, Artificial Sequence
  synthesized KDM2B peptide
VDWVDNMWPQHLKEK

SEQ ID NO: 41 - protein, 15 aa, Artificial Sequence
  synthesized KDM2B peptide
NMWPQHLKEKQTEAT

SEQ ID NO: 42 - protein, 15 aa, Artificial Sequence
  synthesized KDM2B peptide
HLKEKQTEATNAIAE

SEQ ID NO: 43 - protein, 15 aa, Artificial Sequence
  synthesized KDM2B peptide
QTEATNAIAEMKYPK

SEQ ID NO: 44 - protein, 15 aa, Artificial Sequence
  synthesized KDM2B peptide
NAIAEMKYPKVKKYC

SEQ ID NO: 45 - protein, 15 aa, Artificial Sequence
  synthesized KDM2B peptide
MKYPKVKKYCLMSVK

SEQ ID NO: 46 - protein, 15 aa, Artificial Sequence
  synthesized KDM2B peptide
VKKYCLMSVKGCFTD

SEQ ID NO: 47 - protein, 15 aa, Artificial Sequence
  synthesized KDM2B peptide
LMSVKGCFTDFHIDF

SEQ ID NO: 48 - protein, 15 aa, Artificial Sequence
  synthesized KDM2B peptide
GCFTDFHIDFGGTSV

SEQ ID NO: 49 - protein, 15 aa, Artificial Sequence
  synthesized KDM2B peptide
FHIDFGGTSVWYHVF

SEQ ID NO: 50 - protein, 15 aa, Artificial Sequence
  synthesized KDM2B peptide
GGTSVWYHVFRGGKI

SEQ ID NO: 51 - protein, 15 aa, Artificial Sequence
  synthesized KDM2B peptide
WYHVFRGGKIFWLIP

SEQ ID NO: 52 - protein, 15 aa, Artificial Sequence
  synthesized KDM2B peptide
RGGKIFWLIPPTLHN

SEQ ID NO: 53 - protein, 15 aa, Artificial Sequence
  synthesized KDM2B peptide
FWLIPPTLHNLALYE

SEQ ID NO: 54 - protein, 15 aa, Artificial Sequence
  synthesized KDM2B peptide
PTLHNLALYEEWVLS

SEQ ID NO: 55 - protein, 15 aa, Artificial Sequence
  synthesized KDM2B peptide
LALYEEWVLSGKQSD

SEQ ID NO: 56 - protein, 15 aa, Artificial Sequence
  synthesized KDM2B peptide
EWVLSGKQSDIFLGD

SEQ ID NO: 57 - protein, 15 aa, Artificial Sequence
  synthesized KDM2B peptide
GKQSDIFLGDRVERC

SEQ ID NO: 58 - protein, 15 aa, Artificial Sequence
  synthesized KDM2B peptide
IFLGDRVERCQRIEL

SEQ ID NO: 59 - protein, 15 aa, Artificial Sequence
  synthesized KDM2B peptide
RVERCQRIELKQGYT

SEQ ID NO: 60 - protein, 15 aa, Artificial Sequence
  synthesized KDM2B peptide
QRIELKQGYTFFIPS

SEQ ID NO: 61 - protein, 15 aa, Artificial Sequence
  synthesized KDM2B peptide
KQGYTFFIPSGWIHA

SEQ ID NO: 62 - protein, 15 aa, Artificial Sequence
  synthesized KDM2B peptide
FFIPSGWIHAVYTPV

SEQ ID NO: 63 - protein, 15 aa, Artificial Sequence
  synthesized KDM2B peptide
GWIHAVYTPVDSLVF

SEQ ID NO: 64 - protein, 15 aa, Artificial Sequence
  synthesized KDM2B peptide
VYTPVDSLVFGGNIL

SEQ ID NO: 65 - protein, 15 aa, Artificial Sequence
  synthesized KDM2B peptide
DSLVFGGNILHSFNV

SEQ ID NO: 66 - protein, 15 aa, Artificial Sequence
  synthesized KDM2B peptide
GGNILHSFNVPMQLR

SEQ ID NO: 67 - protein, 15 aa, Artificial Sequence
  synthesized KDM2B peptide
HSFNVPMQLRIYEIE

SEQ ID NO: 68 - protein, 15 aa, Artificial Sequence
  synthesized KDM2B peptide
PMQLRIYEIEDRTRV

SEQ ID NO: 69 - protein, 15 aa, Artificial Sequence
  synthesized KDM2B peptide
IYEIEDRTRVQPKFR

SEQ ID NO: 70 - protein, 15 aa, Artificial Sequence
  synthesized KDM2B peptide
DRTRVQPKFRYPFYY

SEQ ID NO: 71 - protein, 15 aa, Artificial Sequence
  synthesized KDM2B peptide
QPKFRYPFYYEMCWY

SEQ ID NO: 72 - protein, 15 aa, Artificial Sequence
  synthesized KDM2B peptide
YPFYYEMCWYVLERY

SEQ ID NO: 73 - protein, 15 aa, Artificial Sequence
  synthesized KDM2B peptide
EMCWYVLERYVYCVT

SEQ ID NO: 74 - protein, 15 aa, Artificial Sequence
  synthesized KDM2B peptide
VLERYVYCVTQRSHL

SEQ ID NO: 75 - protein, 15 aa, Artificial Sequence
  synthesized KDM2B peptide
VYCVTQRSHLTQEYQ

SEQ ID NO: 76 - protein, 15 aa, Artificial Sequence
  synthesized KDM2B peptide
QRSHLTQEYQRESML

SEQ ID NO: 77 - protein, 15 aa, Artificial Sequence
  synthesized KDM2B peptide
TQEYQRESMLIDAPR

SEQ ID NO: 78 - protein, 15 aa, Artificial Sequence
  synthesized KDM2B peptide
RESMLIDAPRKPSID

SEQ ID NO: 79 - protein, 15 aa, Artificial Sequence
  synthesized KDM2B peptide
IDAPRKPSIDGFSSD

SEQ ID NO: 80 - protein, 15 aa, Artificial Sequence
  synthesized KDM2B peptide
KPSIDGFSSDSWLEM

SEQ ID NO: 81 - protein, 15 aa, Artificial Sequence
  synthesized KDM2B peptide
GFSSDSWLEMEEEAC

SEQ ID NO: 82 - protein, 15 aa, Artificial Sequence
  synthesized KDM2B peptide
SWLEMEEEACDQQPQ

SEQ ID NO: 83 - protein, 15 aa, Artificial Sequence
  synthesized KDM2B peptide
EEEACDQQPQEEEEK

SEQ ID NO: 84 - protein, 15 aa, Artificial Sequence
  synthesized KDM2B peptide
DQQPQEEEEKDEEGE

SEQ ID NO: 85 - protein, 15 aa, Artificial Sequence
  synthesized KDM2B peptide
EEEEKDEEGEGRDRA

SEQ ID NO: 86 - protein, 15 aa, Artificial Sequence
  synthesized KDM2B peptide
DEEGEGRDRAPKPPT

SEQ ID NO: 87 - protein, 15 aa, Artificial Sequence
  synthesized KDM2B peptide
GRDRAPKPPTDGSTS

SEQ ID NO: 88 - protein, 15 aa, Artificial Sequence
  synthesized KDM2B peptide
PKPPTDGSTSPTSTP

SEQ ID NO: 89 - protein, 15 aa, Artificial Sequence
  synthesized KDM2B peptide
DGSTSPTSTPSEDQE

SEQ ID NO: 90 - protein, 15 aa, Artificial Sequence
  synthesized KDM2B peptide
PTSTPSEDQEALGKK

SEQ ID NO: 91 - protein, 15 aa, Artificial Sequence
  synthesized KDM2B peptide
SEDQEALGKKPKAPA

SEQ ID NO: 92 - protein, 15 aa, Artificial Sequence
  synthesized KDM2B peptide
ALGKKPKAPALRFLK

SEQ ID NO: 93 - protein, 15 aa, Artificial Sequence
  synthesized KDM2B peptide
PKAPALRFLKRTLSN

SEQ ID NO: 94 - protein, 15 aa, Artificial Sequence
  synthesized KDM2B peptide
LRFLKRTLSNESEES

SEQ ID NO: 95 - protein, 15 aa, Artificial Sequence
  synthesized KDM2B peptide
RTLSNESEESVKSTT

SEQ ID NO: 96 - protein, 15 aa, Artificial Sequence
  synthesized KDM2B peptide
ESEESVKSTTLAVDY

SEQ ID NO: 97 - protein, 15 aa, Artificial Sequence
  synthesized KDM2B peptide
VKSTTLAVDYPKTPT

SEQ ID NO: 98 - protein, 15 aa, Artificial Sequence
  synthesized KDM2B peptide
LAVDYPKTPTGSPAT

SEQ ID NO: 99 - protein, 15 aa, Artificial Sequence
  synthesized KDM2B peptide
PKTPTGSPATEVSAK

SEQ ID NO: 100 - protein, 15 aa, Artificial Sequence
  synthesized KDM2B peptide
GSPATEVSAKWTHLT

SEQ ID NO: 101 - protein, 15 aa, Artificial Sequence
  synthesized KDM2B peptide
EVSAKWTHLTEFELK

SEQ ID NO: 102 - protein, 15 aa, Artificial Sequence
  synthesized KDM2B peptide
WTHLTEFELKGLKAL

SEQ ID NO: 103 - protein, 15 aa, Artificial Sequence
  synthesized KDM2B peptide
EFELKGLKALVEKLE

SEQ ID NO: 104 - protein, 15 aa, Artificial Sequence
  synthesized KDM2B peptide
GLKALVEKLESLPEN

SEQ ID NO: 105 - protein, 15 aa, Artificial Sequence
  synthesized KDM2B peptide
VEKLESLPENKKCVP

SEQ ID NO: 106 - protein, 15 aa, Artificial Sequence
  synthesized KDM2B peptide
SLPENKKCVPEGIED

SEQ ID NO: 107 - protein, 15 aa, Artificial Sequence
  synthesized KDM2B peptide
KKCVPEGIEDPQALL

SEQ ID NO: 108 - protein, 15 aa, Artificial Sequence
  synthesized KDM2B peptide
EGIEDPQALLEGVKN

SEQ ID NO: 109 - protein, 15 aa, Artificial Sequence
  synthesized KDM2B peptide
PQALLEGVKNVLKEH

SEQ ID NO: 110 - protein, 15 aa, Artificial Sequence
  synthesized KDM2B peptide
EGVKNVLKEHADDDP

SEQ ID NO: 111 - protein, 15 aa, Artificial Sequence
  synthesized KDM2B peptide
VLKEHADDDPSLAIT

SEQ ID NO: 112 - protein, 15 aa, Artificial Sequence
  synthesized KDM2B peptide
ADDDPSLAITGVPVV

SEQ ID NO: 113 - protein, 15 aa, Artificial Sequence
  synthesized KDM2B peptide
SLAITGVPVVTWPKK

SEQ ID NO: 114 - protein, 15 aa, Artificial Sequence
  synthesized KDM2B peptide
GVPVVTWPKKTPKNR

SEQ ID NO: 115 - protein, 15 aa, Artificial Sequence
  synthesized KDM2B peptide
TWPKKTPKNRAVGRP

SEQ ID NO: 116 - protein, 15 aa, Artificial Sequence
  synthesized KDM2B peptide
TPKNRAVGRPKGKLG

SEQ ID NO: 117 - protein, 15 aa, Artificial Sequence
  synthesized KDM2B peptide
AVGRPKGKLGPASAV

SEQ ID NO: 118 - protein, 15 aa, Artificial Sequence
  synthesized KDM2B peptide
KGKLGPASAVKLAAN

SEQ ID NO: 119 - protein, 15 aa, Artificial Sequence
  synthesized KDM2B peptide
PASAVKLAANRTTAG

SEQ ID NO: 120 - protein, 15 aa, Artificial Sequence
  synthesized KDM2B peptide
KLAANRTTAGARRRR

SEQ ID NO: 121 - protein, 15 aa, Artificial Sequence
  synthesized KDM2B peptide
RTTAGARRRRTRCRK

SEQ ID NO: 122 - protein, 15 aa, Artificial Sequence
  synthesized KDM2B peptide
ARRRRTRCRKCEACL

SEQ ID NO: 123 - protein, 15 aa, Artificial Sequence
  synthesized KDM2B peptide
TRCRKCEACLRTECG

SEQ ID NO: 124 - protein, 15 aa, Artificial Sequence
  synthesized KDM2B peptide
CEACLRTECGECHFC

SEQ ID NO: 125 - protein, 15 aa, Artificial Sequence
  synthesized KDM2B peptide
RTECGECHFCKDMKK

SEQ ID NO: 126 - protein, 15 aa, Artificial Sequence
  synthesized KDM2B peptide
ECHFCKDMKKFGGPG

SEQ ID NO: 127 - protein, 15 aa, Artificial Sequence
  synthesized KDM2B peptide
KDMKKFGGPGRMKQS

SEQ ID NO: 128 - protein, 15 aa, Artificial Sequence
  synthesized KDM2B peptide
FGGPGRMKQSCIMRQ

SEQ ID NO: 129 - protein, 15 aa, Artificial Sequence
  synthesized KDM2B peptide
RMKQSCIMRQCIAPV

SEQ ID NO: 130 - protein, 15 aa, Artificial Sequence
  synthesized KDM2B peptide
CIMRQCIAPVLPHTA

SEQ ID NO: 131 - protein, 15 aa, Artificial Sequence
  synthesized KDM2B peptide
CIAPVLPHTAVCLVC

SEQ ID NO: 132 - protein, 15 aa, Artificial Sequence
  synthesized KDM2B peptide
LPHTAVCLVCGEAGK

SEQ ID NO: 133 - protein, 15 aa, Artificial Sequence
  synthesized KDM2B peptide
VCLVCGEAGKEDTVE

SEQ ID NO: 134 - protein, 15 aa, Artificial Sequence
  synthesized KDM2B peptide
GEAGKEDTVEEEEGK

SEQ ID NO: 135 - protein, 15 aa, Artificial Sequence
  synthesized KDM2B peptide
EDTVEEEEGKFNLML

SEQ ID NO: 136 - protein, 15 aa, Artificial Sequence
  synthesized KDM2B peptide
EEEGKFNLMLMECSI

SEQ ID NO: 137 - protein, 15 aa, Artificial Sequence
  synthesized KDM2B peptide
FNLMLMECSICNEII

SEQ ID NO: 138 - protein, 15 aa, Artificial Sequence
  synthesized KDM2B peptide
MECSICNEIIHPGCL

SEQ ID NO: 139 - protein, 15 aa, Artificial Sequence
  synthesized KDM2B peptide
CNEIIHPGCLKIKES

SEQ ID NO: 140 - protein, 15 aa, Artificial Sequence
  synthesized KDM2B peptide
HPGCLKIKESEGVVN

SEQ ID NO: 141 - protein, 15 aa, Artificial Sequence
  synthesized KDM2B peptide
KIKESEGVVNDELPN

SEQ ID NO: 142 - protein, 15 aa, Artificial Sequence
  synthesized KDM2B peptide
EGVVNDELPNCWECP

SEQ ID NO: 143 - protein, 15 aa, Artificial Sequence
  synthesized KDM2B peptide
DELPNCWECPKCNHA

SEQ ID NO: 144 - protein, 15 aa, Artificial Sequence
  synthesized KDM2B peptide
CWECPKCNHAGKTGK

SEQ ID NO: 145 - protein, 15 aa, Artificial Sequence
  synthesized KDM2B peptide
KCNHAGKTGKQKRGP

SEQ ID NO: 146 - protein, 15 aa, Artificial Sequence
  synthesized KDM2B peptide
GKTGKQKRGPGFKYA

SEQ ID NO: 147 - protein, 15 aa, Artificial Sequence
  synthesized KDM2B peptide
QKRGPGFKYASNLPG

SEQ ID NO: 148 - protein, 15 aa, Artificial Sequence
  synthesized KDM2B peptide
GFKYASNLPGSLLKE

SEQ ID NO: 149 - protein, 15 aa, Artificial Sequence
  synthesized KDM2B peptide
SNLPGSLLKEQKMNR

SEQ ID NO: 150 - protein, 15 aa, Artificial Sequence
  synthesized KDM2B peptide
SLLKEQKMNRDNKEG

SEQ ID NO: 151 - protein, 15 aa, Artificial Sequence
  synthesized KDM2B peptide
QKMNRDNKEGQEPAK

SEQ ID NO: 152 - protein, 15 aa, Artificial Sequence
  synthesized KDM2B peptide
DNKEGQEPAKRRSEC

SEQ ID NO: 153 - protein, 15 aa, Artificial Sequence
  synthesized KDM2B peptide
QEPAKRRSECEEAPR

SEQ ID NO: 154 - protein, 15 aa, Artificial Sequence
  synthesized KDM2B peptide
RRSECEEAPRRRSDE

SEQ ID NO: 155 - protein, 15 aa, Artificial Sequence
  synthesized KDM2B peptide
EEAPRRRSDEHSKKV

SEQ ID NO: 156 - protein, 15 aa, Artificial Sequence
  synthesized KDM2B peptide
RRSDEHSKKVPPDGL

SEQ ID NO: 157 - protein, 15 aa, Artificial Sequence
  synthesized KDM2B peptide
HSKKVPPDGLLRRKS

SEQ ID NO: 158 - protein, 15 aa, Artificial Sequence
  synthesized KDM2B peptide
PPDGLLRRKSDDVHL

SEQ ID NO: 159 - protein, 15 aa, Artificial Sequence
  synthesized KDM2B peptide
LRRKSDDVHLRKKRK

SEQ ID NO: 160 - protein, 15 aa, Artificial Sequence
  synthesized KDM2B peptide
DDVHLRKKRKYEKPQ

SEQ ID NO: 161 - protein, 15 aa, Artificial Sequence
  synthesized KDM2B peptide
RKKRKYEKPQELSGR

SEQ ID NO: 162 - protein, 15 aa, Artificial Sequence
  synthesized KDM2B peptide
YEKPQELSGRKRASS

SEQ ID NO: 163 - protein, 15 aa, Artificial Sequence
  synthesized KDM2B peptide
ELSGRKRASSLQTSP

SEQ ID NO: 164 - protein, 15 aa, Artificial Sequence
  synthesized KDM2B peptide
KRASSLQTSPGSSSH

SEQ ID NO: 165 - protein, 15 aa, Artificial Sequence
  synthesized KDM2B peptide
LQTSPGSSSHLSPRP

SEQ ID NO: 166 - protein, 15 aa, Artificial Sequence
  synthesized KDM2B peptide
GSSSHLSPRPPLGSS

SEQ ID NO: 167 - protein, 15 aa, Artificial Sequence
  synthesized KDM2B peptide
LSPRPPLGSSLSPWW

SEQ ID NO: 168 - protein, 15 aa, Artificial Sequence
  synthesized KDM2B peptide
PLGSSLSPWWRSSLT

SEQ ID NO: 169 - protein, 15 aa, Artificial Sequence
  synthesized KDM2B peptide
LSPWWRSSLTYFQQQ

SEQ ID NO: 170 - protein, 15 aa, Artificial Sequence
  synthesized KDM2B peptide
RSSLTYFQQQLKPGK

SEQ ID NO: 171 - protein, 15 aa, Artificial Sequence
  synthesized KDM2B peptide
YFQQQLKPGKEDKLF

SEQ ID NO: 172 - protein, 15 aa, Artificial Sequence
  synthesized KDM2B peptide
LKPGKEDKLFRKKRR

SEQ ID NO: 173 - protein, 15 aa, Artificial Sequence
  synthesized KDM2B peptide
EDKLFRKKRRSWKNA

SEQ ID NO: 174 - protein, 15 aa, Artificial Sequence
  synthesized KDM2B peptide
RKKRRSWKNAEDRMA

SEQ ID NO: 175 - protein, 15 aa, Artificial Sequence
  synthesized KDM2B peptide
SWKNAEDRMALANKP

SEQ ID NO: 176 - protein, 15 aa, Artificial Sequence
  synthesized KDM2B peptide
EDRMALANKPLRRFK

SEQ ID NO: 177 - protein, 15 aa, Artificial Sequence
  synthesized KDM2B peptide
LANKPLRRFKQEPED

SEQ ID NO: 178 - protein, 15 aa, Artificial Sequence
  synthesized KDM2B peptide
LRRFKQEPEDELPEA

SEQ ID NO: 179 - protein, 15 aa, Artificial Sequence
  synthesized KDM2B peptide
QEPEDELPEAPPKTR

SEQ ID NO: 180 - protein, 15 aa, Artificial Sequence
  synthesized KDM2B peptide
ELPEAPPKTRESDHS

SEQ ID NO: 181 - protein, 15 aa, Artificial Sequence
  synthesized KDM2B peptide
PPKTRESDHSRSSSP

SEQ ID NO: 182 - protein, 15 aa, Artificial Sequence
  synthesized KDM2B peptide
ESDHSRSSSPTAGPS

SEQ ID NO: 183 - protein, 15 aa, Artificial Sequence
  synthesized KDM2B peptide
RSSSPTAGPSTEGAE

SEQ ID NO: 184 - protein, 15 aa, Artificial Sequence
  synthesized KDM2B peptide
TAGPSTEGAEGPEEK

SEQ ID NO: 185 - protein, 15 aa, Artificial Sequence
  synthesized KDM2B peptide
TEGAEGPEEKKKVKM

SEQ ID NO: 186 - protein, 15 aa, Artificial Sequence
  synthesized KDM2B peptide
GPEEKKKVKMRRKRR

SEQ ID NO: 187 - protein, 15 aa, Artificial Sequence
  synthesized KDM2B peptide
KKVKMRRKRRLPNKE

SEQ ID NO: 188 - protein, 15 aa, Artificial Sequence
  synthesized KDM2B peptide
RRKRRLPNKELSREL

SEQ ID NO: 189 - protein, 15 aa, Artificial Sequence
  synthesized KDM2B peptide
LPNKELSRELSKELN

SEQ ID NO: 190 - protein, 15 aa, Artificial Sequence
  synthesized KDM2B peptide
LSRELSKELNHEIQR

SEQ ID NO: 191 - protein, 15 aa, Artificial Sequence
  synthesized KDM2B peptide
SKELNHEIQRTENSL

SEQ ID NO: 192 - protein, 15 aa, Artificial Sequence
  synthesized KDM2B peptide
HEIQRTENSLANENQ

SEQ ID NO: 193 - protein, 15 aa, Artificial Sequence
  synthesized KDM2B peptide
TENSLANENQQPIKS

SEQ ID NO: 194 - protein, 15 aa, Artificial Sequence
  synthesized KDM2B peptide
ANENQQPIKSEPESE

SEQ ID NO: 195 - protein, 15 aa, Artificial Sequence
  synthesized KDM2B peptide
QPIKSEPESEGEEPK

SEQ ID NO: 196 - protein, 15 aa, Artificial Sequence
  synthesized KDM2B peptide
EPESEGEEPKRPPGI

SEQ ID NO: 197 - protein, 15 aa, Artificial Sequence
  synthesized KDM2B peptide
GEEPKRPPGICERPH

SEQ ID NO: 198 - protein, 15 aa, Artificial Sequence
  synthesized KDM2B peptide
RPPGICERPHRFSKG

SEQ ID NO: 199 - protein, 15 aa, Artificial Sequence
  synthesized KDM2B peptide
CERPHRFSKGLNGTP

SEQ ID NO: 200 - protein, 15 aa, Artificial Sequence
  synthesized KDM2B peptide
RFSKGLNGTPRELRH

SEQ ID NO: 201 - protein, 15 aa, Artificial Sequence
  synthesized KDM2B peptide
LNGTPRELRHQLGPS

SEQ ID NO: 202 - protein, 15 aa, Artificial Sequence
  synthesized KDM2B peptide
RELRHQLGPSLRSPP

SEQ ID NO: 203 - protein, 15 aa, Artificial Sequence
  synthesized KDM2B peptide
QLGPSLRSPPRVISR

SEQ ID NO: 204 - protein, 15 aa, Artificial Sequence
  synthesized KDM2B peptide
LRSPPRVISRPPPSV

SEQ ID NO: 205 - protein, 15 aa, Artificial Sequence
  synthesized KDM2B peptide
RVISRPPPSVSPPKC

SEQ ID NO: 206 - protein, 15 aa, Artificial Sequence
  synthesized KDM2B peptide
PPPSVSPPKCIQMER

SEQ ID NO: 207 - protein, 15 aa, Artificial Sequence
  synthesized KDM2B peptide
SPPKCIQMERHVIRP

SEQ ID NO: 208 - protein, 15 aa, Artificial Sequence
  synthesized KDM2B peptide
IQMERHVIRPPPISP

SEQ ID NO: 209 - protein, 15 aa, Artificial Sequence
  synthesized KDM2B peptide
HVIRPPPISPPPDSL

SEQ ID NO: 210 - protein, 15 aa, Artificial Sequence
  synthesized KDM2B peptide
PPISPPPDSLPLDDG

SEQ ID NO: 211 - protein, 15 aa, Artificial Sequence
  synthesized KDM2B peptide
PPDSLPLDDGAAHVM

SEQ ID NO: 212 - protein, 15 aa, Artificial Sequence
  synthesized KDM2B peptide
PLDDGAAHVMHREVW

SEQ ID NO: 213 - protein, 15 aa, Artificial Sequence
  synthesized KDM2B peptide
AAHVMHREVWMAVFS

SEQ ID NO: 214 - protein, 15 aa, Artificial Sequence
  synthesized KDM2B peptide
HREVWMAVFSYLSHQ

SEQ ID NO: 215 - protein, 15 aa, Artificial Sequence
  synthesized KDM2B peptide
MAVFSYLSHQDLCVC

SEQ ID NO: 216 - protein, 15 aa, Artificial Sequence
  synthesized KDM2B peptide
YLSHQDLCVCMRVCR

SEQ ID NO: 217 - protein, 15 aa, Artificial Sequence
  synthesized KDM2B peptide
DLCVCMRVCRTWNRW

SEQ ID NO: 218 - protein, 15 aa, Artificial Sequence
  synthesized KDM2B peptide
MRVCRTWNRWCCDKR

SEQ ID NO: 219 - protein, 15 aa, Artificial Sequence
  synthesized KDM2B peptide
TWNRWCCDKRLWTRI

SEQ ID NO: 220 - protein, 15 aa, Artificial Sequence
  synthesized KDM2B peptide
CCDKRLWTRIDLNHC

SEQ ID NO: 221 - protein, 15 aa, Artificial Sequence
  synthesized KDM2B peptide
LWTRIDLNHCKSITP

SEQ ID NO: 222 - protein, 15 aa, Artificial Sequence
  synthesized KDM2B peptide
DLNHCKSITPLMLSG

SEQ ID NO: 223 - protein, 15 aa, Artificial Sequence
  synthesized KDM2B peptide
KSITPLMLSGIIRRQ

SEQ ID NO: 224 - protein, 15 aa, Artificial Sequence
  synthesized KDM2B peptide
LMLSGIIRRQPVSLD

SEQ ID NO: 225 - protein, 15 aa, Artificial Sequence
  synthesized KDM2B peptide
IIRRQPVSLDLSWTN

SEQ ID NO: 226 - protein, 15 aa, Artificial Sequence
  synthesized KDM2B peptide
PVSLDLSWTNISKKQ

SEQ ID NO: 227 - protein, 15 aa, Artificial Sequence
  synthesized KDM2B peptide
LSWTNISKKQLSWLI

SEQ ID NO: 228 - protein, 15 aa, Artificial Sequence
  synthesized KDM2B peptide
ISKKQLSWLINRLPG

SEQ ID NO: 229 - protein, 15 aa, Artificial Sequence
  synthesized KDM2B peptide
LSWLINRLPGLRDLV

SEQ ID NO: 230 - protein, 15 aa, Artificial Sequence
  synthesized KDM2B peptide
NRLPGLRDLVLSGCS

SEQ ID NO: 231 - protein, 15 aa, Artificial Sequence
  synthesized KDM2B peptide
LRDLVLSGCSWIAVS

SEQ ID NO: 232 - protein, 15 aa, Artificial Sequence
  synthesized KDM2B peptide
LSGCSWIAVSALCSS

SEQ ID NO: 233 - protein, 15 aa, Artificial Sequence
  synthesized KDM2B peptide
WIAVSALCSSSCPLL

SEQ ID NO: 234 - protein, 15 aa, Artificial Sequence
  synthesized KDM2B peptide
ALCSSSCPLLRTLDV

SEQ ID NO: 235 - protein, 15 aa, Artificial Sequence
  synthesized KDM2B peptide
SCPLLRTLDVQWVEG

SEQ ID NO: 236 - protein, 15 aa, Artificial Sequence
  synthesized KDM2B peptide
RTLDVQWVEGLKDAQ

SEQ ID NO: 237 - protein, 15 aa, Artificial Sequence
  synthesized KDM2B peptide
QWVEGLKDAQMRDLL

SEQ ID NO: 238 - protein, 15 aa, Artificial Sequence
  synthesized KDM2B peptide
LKDAQMRDLLSPPTD

SEQ ID NO: 239 - protein, 15 aa, Artificial Sequence
  synthesized KDM2B peptide
MRDLLSPPTDNRPGQ

SEQ ID NO: 240 - protein, 15 aa, Artificial Sequence
  synthesized KDM2B peptide
SPPTDNRPGQMDNRS

SEQ ID NO: 241 - protein, 15 aa, Artificial Sequence
  synthesized KDM2B peptide
NRPGQMDNRSKLRNI

SEQ ID NO: 242 - protein, 15 aa, Artificial Sequence
  synthesized KDM2B peptide
MDNRSKLRNIVELRL

SEQ ID NO: 243 - protein, 15 aa, Artificial Sequence
  synthesized KDM2B peptide
KLRNIVELRLAGLDI

SEQ ID NO: 244 - protein, 15 aa, Artificial Sequence
  synthesized KDM2B peptide
VELRLAGLDITDASL

SEQ ID NO: 245 - protein, 15 aa, Artificial Sequence
  synthesized KDM2B peptide
AGLDITDASLRLIIR

SEQ ID NO: 246 - protein, 15 aa, Artificial Sequence
  synthesized KDM2B peptide
TDASLRLIIRHMPLL

SEQ ID NO: 247 - protein, 15 aa, Artificial Sequence
  synthesized KDM2B peptide
RLIIRHMPLLSKLHL

SEQ ID NO: 248 - protein, 15 aa, Artificial Sequence
  synthesized KDM2B peptide
HMPLLSKLHLSYCNH

SEQ ID NO: 249 - protein, 15 aa, Artificial Sequence
  synthesized KDM2B peptide
SKLHLSYCNHVTDQS

SEQ ID NO: 250 - protein, 15 aa, Artificial Sequence
  synthesized KDM2B peptide
SYCNHVTDQSINLLT

SEQ ID NO: 251 - protein, 15 aa, Artificial Sequence
  synthesized KDM2B peptide
VTDQSINLLTAVGTT

SEQ ID NO: 252 - protein, 15 aa, Artificial Sequence
  synthesized KDM2B peptide
INLLTAVGTTTRDSL

SEQ ID NO: 253 - protein, 15 aa, Artificial Sequence
  synthesized KDM2B peptide
AVGTTTRDSLTEINL

SEQ ID NO: 254 - protein, 15 aa, Artificial Sequence
  synthesized KDM2B peptide
TRDSLTEINLSDCNK

SEQ ID NO: 255 - protein, 15 aa, Artificial Sequence
  synthesized KDM2B peptide
TEINLSDCNKVTDQC

SEQ ID NO: 256 - protein, 15 aa, Artificial Sequence
  synthesized KDM2B peptide
SDCNKVTDQCLSFFK

SEQ ID NO: 257 - protein, 15 aa, Artificial Sequence
  synthesized KDM2B peptide
VTDQCLSFFKRCGNI

SEQ ID NO: 258 - protein, 15 aa, Artificial Sequence
  synthesized KDM2B peptide
LSFFKRCGNICHIDL

SEQ ID NO: 259 - protein, 15 aa, Artificial Sequence
  synthesized KDM2B peptide
RCGNICHIDLRYCKQ

SEQ ID NO: 260 - protein, 15 aa, Artificial Sequence
  synthesized KDM2B peptide
CHIDLRYCKQVTKEG

SEQ ID NO: 261 - protein, 15 aa, Artificial Sequence
  synthesized KDM2B peptide
RYCKQVTKEGCEQFI

SEQ ID NO: 262 - protein, 15 aa, Artificial Sequence
  synthesized KDM2B peptide
VTKEGCEQFIAEMSV

SEQ ID NO: 263 - protein, 15 aa, Artificial Sequence
  synthesized KDM2B peptide
CEQFIAEMSVSVQFG

SEQ ID NO: 264 - protein, 15 aa, Artificial Sequence
  synthesized KDM2B peptide
AEMSVSVQFGQVEEK

SEQ ID NO: 265 - protein, 15 aa, Artificial Sequence
  synthesized KDM2B peptide
SVQFGQVEEKLLQKL

SEQ ID NO: 266 - protein, 15 aa, Artificial Sequence
  synthesized KDM2B peptide
VQFGQVEEKLLQKLS

SEQ ID NO: 267 - protein, 20 aa, Artificial Sequence
  synthesized KDM2B peptide
EEEACDQQPQEEEEKDEEGE

SEQ ID NO: 268 - protein, 20 aa, Artificial Sequence
  synthesized KDM2B peptide
VKKYCLMSVKGCFTDFHIDF

SEQ ID NO: 269 - protein, 20 aa, Artificial Sequence
  synthesized KDM2B peptide
ARRRRTRCRKCEACLRTECG

SEQ ID NO: 270 - protein, 20 aa, Artificial Sequence
  synthesized KDM2B peptide
CIAPVLPHTAVCLVCGEAGK

SEQ ID NO: 271 - protein, 30 aa, Artificial Sequence
  synthesized KDM2B peptide
CNEIIHPGCLKIKESEGVVNDELPNCWECP

SEQ ID NO: 272 - protein, 25 aa, Artificial Sequence
  synthesized KDM2B peptide
QKMNRDNKEGQEPAKRRSECEEAPR

SEQ ID NO: 273 - protein, 25 aa, Artificial Sequence
  synthesized KDM2B peptide
LRDLVLSGCSWIAVSALCSSSCPLL

SEQ ID NO: 274 - DNA, 22 bp, Artificial Sequence
  PCR primer
cggaccaatacgaccaaatccg

SEQ ID NO: 275 - DNA, 20 bp, Artificial Sequence
  PCR primer
agccacatcgctcagacacc

SEQ ID NO: 276 - DNA, 21 bp, Artificial Sequence
  PCR primer
tgtcccagtttgtgcgttact

SEQ ID NO: 277 - DNA, 19 bp, Artificial Sequence
  PCR primer
ggacgcttgaccaagtgct

SEQ ID NO: 278 - DNA, 24 bp, Artificial Sequence
  PCR primer
cgggacctggaggacttctacccg

SEQ ID NO: 279 - DNA, 21 bp, Artificial Sequence
  PCR primer
accatgtgcccatccagagtc

SEQ ID NO: 280 - DNA, 26 bp, Artificial Sequence
  PCR primer
cgaagtcaatggtttcctccacttcg

SEQ ID NO: 281 - DNA, 22 bp, Artificial Sequence
  PCR primer
ccgctccttcccgtcctactac

SEQ ID NO: 282 - DNA, 26 bp, Artificial Sequence
  PCR primer
cgactgacccctactcctaccagtcg

SEQ ID NO: 283 - DNA, 21 bp, Artificial Sequence
  PCR primer
tggaagacatgggagctgtcc

SEQ ID NO: 284 - DNA, 21 bp, Artificial Sequence
  PCR primer
ccgagctgtgaaggtgtttga

SEQ ID NO: 285 - DNA, 18 bp, Artificial Sequence
  PCR primer
cgggccgggtctctagat

SEQ ID NO: 286 - protein, 11 aa, Artificial Sequence
  cell-penetrating peptide
YGRKKRRQRRR

SEQ ID NO: 287 - DNA, 23 bp, Artificial Sequence
  KDM2B shRNA
attttgacggggtggataatctg